(19) United States
(12) Patent Application Publication
(10) Pub. No.: US 2019/0298674 A1
(43) Pub. Date: Oct. 3, 2019

(54) USE OF (1S,3S)-3-AMINO-4-(DIFLUOROMETHYLIDENE) CYCLOPENTANE-1-CARBOXYLIC ACID AND (S)-3-AMINO-4-(DIFLUOROMETHYLENYL)CYCLOPENT-1-ENE-1-CARBOXYLIC ACID IN THE TREATMENT OF EYE DISORDERS

(71) Applicant: Ovid Therapeutics, Inc., New York, NY (US)

(72) Inventor: Matthew During, Weston, CT (US)

(21) Appl. No.: 16/368,907

(22) Filed: Mar. 29, 2019

Related U.S. Application Data
(60) Provisional application No. 62/650,004, filed on Mar. 29, 2018.

(51) Int. Cl. A61K 31/196; A61P 27/02

(52) U.S. Cl. CPC A61K 31/196; A61P 27/02

(57) ABSTRACT

Methods of treating eye disorders with (S)-3-amino-4-(difluoromethylenyl)cyclopent-1-ene-1-carboxylic acid are provided. Methods of treating eye disorders with (1S,3S)-3-amino-4-(difluoromethylidene) cyclopentane-1-carboxylic acid or a pharmaceutically acceptable salt thereof are provided. Also provided are therapeutic compositions that may be used to improve one or more symptoms of eye disorders.

### CROSS-REFERENCE TO RELATED APPLICATION

This application claims benefit and priority to U.S. Provisional Application No. 62/650,004, filed Mar. 29, 2018, which is incorporated herein by reference in its entirety.

### TECHNICAL FIELD

Methods of treating eye disorders with (1S,3S)-3-amino-4-(difluoromethylidene) cyclopentane-1-carboxylic acid, (S)-3-amino-4-(difluoromethylenyl) cyclopent-1-ene-1-carboxylic acid or pharmaceutically acceptable salts of either of the foregoing.

### BACKGROUND

The retina is a complex tissue in the back of the eye that contains specialized photoreceptor cells called rods and cones. The photoreceptors connect to a network of nerve cells for the local processing of visual information. This information is sent to the brain for decoding into a visual image. The adjacent retinal pigment epithelium (RPE) supports many of the retina's metabolic functions. Retinal degeneration may be caused by a variety of disorders and is a retinopathy which involves deterioration of the retina caused by the progressive death of its cells. Symptoms of retinal degeneration can be impaired vision, night blindness, retinal detachment, light sensitivity, tunnel vision, and loss of peripheral vision to total loss of vision. Retinal dystrophy is a term applied to a wide range of eye disorders, i.e., “dystrophy” means a condition that a person is born with; “retinal” means relating to the retina.

Many disorders are known to affect the eye, including macular degeneration, also known as age-related macular degeneration (AMD or ARMD), juvenile macular degeneration, retinal degeneration, glaucoma, retinal dystrophy, Doyne honeycomb retinal dystrophy, Stargardt disease, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, Retinoblastoma, Usher syndrome, Behçet's disease, Achromatopsia 2, Acute posterior multifocal placoid pigment epitheliopathy (APMPPE), Acute zonal occult outer retinopathy (AZOOR), Adult-onset vitelliform macular dystrophy (AVMD), Ocular albinism with late-onset sensorineural deafness (OASD), Alström syndrome, Anterior ischemic optic neuropathy, corneal amyloidosis, Gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, Birdshot chorioretinopathy, Blue cone monochromatism, central areolar choroidal dystrophy, Choroideremia, Coats disease, Iridocorneal endothelial (ICE) syndrome, Avellino type corneal dystrophy, Schnyder corneal dystrophy, Thiel-Behnke corneal dystrophy, Eales disease, Epithelial basement membrane corneal dystrophy, Fish-eye disease, Fuchs endothelial corneal dystrophy, Goldmann-Favre syndrome, Juvenile retinoschisis, late-onset retinal degeneration, Leber congenital amaurosis, retinitis pigmentosa, Peters anomaly, Punctate inner choroidopathy, Senior Loken syndrome, Snowflake vitreoretinal degeneration, Usher syndrome, Visual snow syndrome, Wagner syndrome, and other inherited retinal degenerations. Each of these can lead to visual loss or complete blindness.

### SUMMARY

Methods of treating eye disorders are provided and, in embodiments, include administering to a subject in need thereof an effective amount of (1S,3S)-3-amino-4-(difluoromethylidene) cyclopentane-1-carboxylic acid or a pharmaceutically acceptable salt thereof. In embodiments, methods of treating an eye disorder include administering (1S,3S)-3-amino-4-(difluoromethylidene) cyclopentane-1-carboxylic acid or a pharmaceutically acceptable salt thereof to a subject in need thereof to provide improvement in one or more symptoms of the eye disorder. In embodiments, methods of treating an eye disorder include administering (1S,3S)-3-amino-4-(difluoromethylidene) cyclopentane-1-carboxylic acid or a pharmaceutically acceptable salt thereof to a subject in need thereof to provide improvement in the eye disorder in the subject the next day after administration. In embodiments, methods of treating an eye disorder include administering to a subject in need thereof an effective amount of (S)-3-amino-4-(difluoromethylenyl)cyclopent-1-ene-1-carboxylic acid or a pharmaceutically acceptable salt thereof. In embodiments, methods of treating an eye disorder include administering (S)-3-amino-4-(difluoromethylenyl)cyclopent-1-ene-1-carboxylic acid or a pharmaceutically acceptable salt thereof to a subject in need thereof to provide improvement in one or more symptoms of the eye disorder. In embodiments, methods of treating an eye disorder include administering (S)-3-amino-4-(difluoromethylenyl)cyclopent-1-ene-1-carboxylic acid or a pharmaceutically acceptable salt thereof to a subject in need thereof to provide improvement in the eye disorder in the subject the next day after administration.

In embodiments, methods of treating an eye disorder include administering: (1 S,3 S)-3-amino-4-(difluoromethylidene)cyclopentane-1-carboxylic acid or a pharmaceutically acceptable salt thereof, and (S)-3-amino-4-(difluoromethylenyl)cyclopent-1-ene-1-carboxylic acid or a pharmaceutically acceptable salt thereof, to a subject in need thereof.

Pharmaceutical compositions including (1 S,3 S)-3-amino-4-(difluoromethylidene)cyclopentane-1-carboxylic acid or a pharmaceutically acceptable salt thereof, and/or (S)-3-amino-4-(difluoromethylenyl)cyclopent-1-ene-1-carboxylic acid or a pharmaceutically acceptable salt thereof are provided.

In embodiments, (1 S,3 S)-3-amino-4-(difluoromethylidene)cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof is administered orally or parenterally. In embodiments, (1 S,3 S)-3-amino-4-(difluoromethylidene)cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof is administered ophthalmically.

### DETAILED DESCRIPTION

Described herein are methods and compositions for treating an eye disorder which include administering to a subject in need thereof an effective amount of (1 S,3 S)-3-amino-4-(difluoromethylidene)cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing.

Described herein are methods of treating an eye disorder with (1S,3S)-3-amino-4-(difluoromethylidene)cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing. Methods of treating an eye disorder are provided and, in embodiments, include administering to a subject in need thereof an effective amount of (1S,3S)-3-amino-4-(difluoromethylidene)cyclopentane-1-carboxylic acid or a pharmaceutically acceptable salt thereof. In embodiments, methods of treating an eye disorder include administering (1 S,3 S)-3-amino-4-(difluoromethylidene)cyclopentane-1-carboxylic acid or a pharmaceutically acceptable salt thereof to a subject in need thereof to provide improvement in one or more symptoms of the eye disorder in the subject. In embodiments, methods of treating an eye disorder include administering (1 S, 3 S)-3-amino-4-(difluoromethylidene)cyclopentane-1-carboxylic acid or a pharmaceutically acceptable salt thereof to a subject in need thereof to provide improvement in the eye disorder in the subject the next day after administration. In embodiments, methods of treating an eye disorder include administering to a subject in need thereof an effective amount of (S)-3-amino-4-(difluoromethylenyl)cyclopent-1-ene-1-carboxylic acid or a pharmaceutically acceptable salt thereof. In embodiments, methods of treating an eye disorder include administering (S)-3-amino-4-(difluoromethylenyl)cyclopent-1-ene-1-carboxylic acid or a pharmaceutically acceptable salt thereof to a subject in need thereof to provide improvement in one or more symptoms of the eye disorder. In embodiments, methods of treating an eye disorder include administering (S)-3-amino-4-(difluoromethylenyl)cyclopent-1-ene-1-carboxylic acid or a pharmaceutically acceptable salt thereof to a subject in need thereof to provide improvement in the eye disorder in the subject the next day after administration. In embodiments, methods of treating an eye disorder include administering (1 S,3 S)-3-amino-4-(difluoromethylidene)cyclopentane-1-carboxylic acid or a pharmaceutically acceptable salt thereof, in combination with (S)-3-amino-4-(difluoromethylenyl)cyclopent-1-ene-1-carboxylic acid or a pharmaceutically acceptable salt thereof, to a subject in need thereof.

The structure of (1 S,3 S)-3-amino-4-(difluoromethylidene)cyclopentane-1-carboxylic acid may be represented as follows:

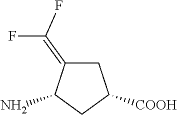 [SMILES: N[C@H]1C[C@@H](C(=O)O)CC1=C(F)F]

The structure of (S)-3-amino-4-(difluoromethylenyl)cyclopent-1-ene-1-carboxylic acid may be represented as follows:

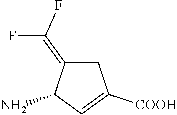 [SMILES: N[C@H]1C=C(C(=O)O)CC1=C(F)F]

Eye disorders suitable for treatment herein include Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy (APMPPE), acute zonal occult outer retinopathy (AZOOR), adult-onset vitelliform macular dystrophy (AVMD), ocular albinism with late-onset sensorineural deafness (OASD), Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome.

Age Related Macular Degeneration (AMD or ARMD) is a common eye condition and a leading cause of vision loss among people age 50 and older. It causes damage to the macula, a small spot near the center of the retina and the part of the eye needed for sharp, central vision. The presence of medium-to-large drusen, which are white or yellow deposits beneath the retina, may indicate AMD. There are three stages of AMD defined in part by the size and number of drusen under the retina: 1) Early AMD which is diagnosed by the presence of medium-sized drusen, which are about the width of an average human hair. People with early AMD typically do not have vision loss. 2) People with intermediate AMD typically have large drusen, pigment changes in the retina, or both. These changes can only be detected during an eye exam. Intermediate AMD may cause some vision loss, but most people will not experience any symptoms. 3) Late AMD. In addition to drusen, people with late AMD have vision loss from damage to the macula. There are two types of late AMD: A) Dry AMD (also called geographic atrophy, atrophic AMD, non-neovascular AMD, or non-exudative AMD), wherein there is a gradual breakdown or thinning of the light-sensitive cells in the macula that convey visual information to the brain and of the supporting tissue beneath the macula (photoreceptors, retinal pigment epithelium, choriocappillaris). These changes cause vision loss. B) Neovascular AMD (also called wet AMD), wherein abnormal blood vessels grow in the choroid layer underneath the retina. These vessels can leak fluid and blood, which may lead to swelling and damage of the macula. The damage may be rapid and severe, unlike the more gradual course of geographic atrophy. It is possible to have both geographic atrophy and neovascular AMD in the same eye, and either condition can appear first.

Juvenile macular degeneration is a series of inherited eye disorders that affects children and young adults. Juvenile macular degeneration is different from age-related macular degeneration, which occurs as part of the body's natural aging process. Juvenile macular degeneration is sometimes called juvenile macular dystrophy. They include Stargardt's disease, Best disease, and juvenile retinoschisis. They can cause central vision loss that often starts in childhood or young adulthood.

Stargardt disease is an inherited disorder of the retina. Stargardt disease is also called Stargardt macular dystrophy, or fundus flavimaculatus. The disease causes progressive damage—or degeneration—of the macula, The disease typically causes vision loss during childhood or adolescence, although in some forms, vision loss may not be noticed until later in adulthood. Vision loss is due to abnormal accumulation of a fatty yellow pigment (lipofuscin) in the cells within the macula. People with Stargardt disease also have problems with night vision, and some have problems with color vision. The signs and symptoms of Stargardt disease typically appear in late childhood to early adulthood and worsen over time. The most common cause is believed to be by mutations in the ABCA4 gene.

Best disease (vitelliform macular dystrophy) (BVMD) is a slowly progressive form of macular degeneration. It usually begins in childhood or adolescence, but age of onset and severity of vision loss can vary. Affected people first have normal vision, followed by decreased central visual acuity and distorted vision (metamorphopsia). Peripheral vision is not affected. BVMD is characterized by atrophy of the retinal pigment epithelium and impaired central visual function.

Adult-onset vitelliform macular dystrophy (AVMD) is an eye disorder that can cause progressive vision loss. AVMD affects an area of the retina called the macula, which is responsible for sharp central vision. The condition causes fatty yellow pigment to accumulate in cells underlying the macula, eventually damaging the cells.

Juvenile retinoschisis is an eye condition characterized by impaired vision that begins in childhood and occurs almost exclusively in males. The condition affects the retina and affects the sharpness of vision. Central vision is more commonly affected. Vision often deteriorates early in life, but then usually becomes stable until late adulthood. A second decline in vision typically occurs in a person's fifties or sixties.

Light induced retinal damage (LIRD) resembles many features of several retinal degenerative diseases, particularly age-related macular degeneration. Photochemical damage is the most common form of LIRD and occurs when light is absorbed by a chromophore and leads to the formation of an electronically excited state of that molecule, which then undergoes either chemical transformation itself and/or interacts with other molecules leading to chemical changes of both interacting molecules. Light damage in the human retina due to excessive exposure to sunlight is known as solar retinopathy. Photochemical damage typically damages the rods and cones of the eye.

Doyne honeycomb retinal dystrophy is a condition that affects the eyes and causes vision loss. It is characterized by small, round, white drusen that accumulate beneath the retinal pigment epithelium (the pigmented layer of the retina). Over time, drusen may grow and come together, creating a honeycomb pattern. It usually begins in early to mid-adulthood, but the age of onset can vary. The degree of vision loss also varies.

Uveitis is the inflammation of the uvea, the pigmented layer that lies between the inner retina and the outer fibrous layer composed of the sclera and cornea. The uvea consists of the iris, the ciliary body and the choroid. The type of uveitis depends on which structure is affected. Iritis (anterior uveitis) affects the front of the eye and is the most common type. Cyclitis (intermediate uveitis) affects the ciliary body. Choroiditis and retinitis (posterior uveitis) affect the back of the eye. Diffuse uveitis (panuveitis) occurs when all layers of the uvea are inflamed. Warning signs often come on suddenly and can get worse quickly. They include eye redness, pain, light sensitivity, floaters and blurred vision. Possible causes of uveitis are infection, injury, or an autoimmune or inflammatory disease.

Scleritis, or inflammation of the sclera, can present as a painful red eye with or without vision loss. Scleritis may be associated with autoimmune disorders, connective tissue disorders and generalized vasculitic abnormalities. Scleritis can also result from an infectious process caused by bacteria including pseudomonas, fungi, mycobacterium, viruses, or parasites. The most common form, anterior scleritis, is defined as scleral inflammation anterior to the extraocular recti muscles. Posterior scleritis is defined as involvement of the sclera posterior to the insertion of the rectus muscles. Anterior scleritis can be subdivided into diffuse, nodular, or necrotizing forms. In the diffuse form, anterior scleral edema is present along with dilation of the deep episcleral vessels. The entire anterior sclera or just a portion may be involved. In nodular disease, a distinct nodule of scleral edema is present. The nodules may be single or multiple in appearance. Necrotizing anterior scleritis is the most severe form of scleritis. Scleritis is characterized by severe pain and extreme scleral tenderness.

Sarcoidosis is a systemic autoinflammatory disease that can affect multiple parts of the body and cause varying levels of inflammation. Ocular sarcoidosis can involve any part of the eye and its adnexal tissues, and may cause uveitis, episcleritis/scleritis, eyelid abnormalities, conjunctival granuloma, optic neuropathy, lacrimal gland enlargement and orbital inflammation. Ocular sarcoidosis can be a “granulomatous” uveitis, i.e., it creates large clumps or collections of inflammatory cells visible on the back of the cornea on exam. Glaucoma and cataracts can be complications from inflammation itself or adverse effects from therapy. Ocular sarcoidosis can manifest itself with blurred vision, photophobia, floaters, redness, and pain from uveitis.

Optic neuritis is inflammation of the optic nerve, which is the nerve that carries visual signals from the eye to the brain. The condition may cause pain and sudden, reduced vision, and/or blurry vision in the affected eye(s). Other early symptoms can be reduced night vision, sensitivity to light (photophobia) and red eyes. Some common causes of optic neuritis are multiple sclerosis and blood clots. Optic neuritis may also be caused by autoimmune disease and diabetes.

Cone-rod dystrophy is a group of inherited eye disorders that affect the light sensitive cells of the retina, i.e., the cones and rods. People with this condition experience vision loss over time as the cones and rods deteriorate. Initial signs and symptoms that usually occur in childhood may include decreased sharpness of vision (visual acuity) and abnormal sensitivity to light (photophobia). These signs are usually followed by blind spots in the central field of vision (scotomas), loss of color perception, night blindness and loss of peripheral vision. Cone-rod dystrophy can be either autosomal dominant, autosomal recessive or X-linked and may be caused by defects in at least 17 different genes.

Macular edema is the build-up of fluid in the macula. Fluid buildup causes the macula to swell and thicken, which distorts vision. Macular edema is typically caused by increased leakage from damaged retinal blood vessels or growth of abnormal blood vessels in the deep retina. Macular edema may also be caused by inflammatory processes. Macular edema may commonly be associated with diabetes. Age-related macular degeneration may also cause macular edema.

Diabetic retinopathy is caused by chronically high blood sugar from diabetes and is associated with damage to the tiny blood vessels in the retina. Diabetic retinopathy can cause blood vessels in the retina to leak fluid or hemorrhage, distorting vision. In its most advanced stage, new abnormal blood vessels proliferate on the surface of the retina, which can lead to scarring and cell loss in the retina. Diabetic retinopathy can progress through four stages: 1) Mild nonproliferative retinopathy in which small areas of balloon-like swelling in the retina's tiny blood vessels, called microaneurysms, occur at this earliest stage of the disease. These microaneurysms may leak fluid into the retina. 2) Moderate nonproliferative retinopathy which, as the disease progresses, blood vessels that nourish the retina may swell and distort. They may also lose their ability to transport blood. Both conditions cause characteristic changes to the appearance of the retina and may contribute to diabetic macula edema. 3) Severe nonproliferative retinopathy in which many more blood vessels are blocked, depriving blood supply to areas of the retina. These areas secrete growth factors that signal the retina to grow new blood vessels. 4) Proliferative diabetic retinopathy (PDR): At this advanced stage, growth factors secreted by the retina trigger the proliferation of new blood vessels, which grow along the inside surface of the retina and into the vitreous gel. The new blood vessels are fragile, which makes them more likely to leak and bleed. Accompanying scar tissue can contract and cause retinal detachment. Retinal detachment can lead to permanent vision loss.

Diabetic macular edema is the build-up of fluid in the macula in diabetics and is associated with diabetic retinopathy. Diabetic retinopathy damages the blood vessels in the retina. Left untreated, these blood vessels begin to build up pressure in the eye and leak fluid, causing diabetic macular edema. Common symptoms of diabetic macular edema are blurry vision, floaters, double vision, and eventually blindness if left untreated.

Corneal ulcer, or ulcerative keratitis, is an inflammatory or more seriously, infective condition of the cornea involving disruption of its epithelial layer with involvement of the corneal stroma. A corneal ulcer is essentially an open sore. Corneal ulcers are extremely painful due to nerve exposure, and can cause tearing, squinting, and vision loss of the eye. There may also be signs of anterior uveitis, such as miosis (small pupil), aqueous flare (protein in the aqueous humour), and redness of the eye. Corneal ulcers can be caused by trauma or by infective microorganisms such as bacteria, fungi, protozoans and viruses.

Optic nerve degeneration aka optic atrophy refers to a group of conditions in which the optic nerve is damaged: some are genetically-based while others are due to trauma, toxins, deficiencies, and inflammation. In optic nerve degeneration the optic nerve is limited in its capacity to transmit accurate information about visual input in the form of electrical impulses to the brain. Symptoms can include blurred vision, decrease in visual acuity, decreases in peripheral vision, decrease in color vision, decrease in contrast sensitivity, and poor constriction of the pupil when exposed to light. Some causes of optic nerve degeneration are glaucoma, diabetes, stroke of the optic nerve aka ischemic optic neuropathy, a tumor pressing on the optic nerve, and optic neuritis.

Choroidal dystrophies are a group of inherited disorders that involve the choroid and can involve the retina. They include choroideremia, gyrate atrophy, central areolar dystophy, diffuse choroidal atrophy, helicoid peripapillary chorioretinal degeneration and pigmented paravenous retinochoroidal atrophy. Choroideremia is a genetic condition that causes vision loss and typically affects males. The first symptom is usually impairment of night vision (night blindness), which can occur in childhood. People with this disorder also experience narrowing of the field of vision (tunnel vision) and decrease in the ability to see details (visual acuity). The vision problems are due to loss of cells in the retina and choroid. Gyrate atrophy, also known as ornithine aminotransferase deficiency, is an autosomal recessive dystrophy caused by mutations in the gene for ornithine aminotransferase. Symptoms include myopia, often appearing in early childhood, leading to night blindness, limited visual field, and posterior subcapsular cataracts. Symptoms of gyrate atrophy are progressive and can lead to complete blindness by age 45 to 65. Central areolar choroidal dystrophy is a hereditary macular disorder, usually presenting between the ages of 30-60, characterized by a large area of atrophy in the center of the macula and the loss or absence of photoreceptors, retinal pigment epithelium and choriocapillaris in this area, resulting in a progressive decrease in visual acuity. Diffuse choroidal dystrophy is an inherited autosomal dominant disorder that affects the choroid and retina. It is similar to central areolar choroidal dystrophy but is characterized by earlier manifestations of the disease by about ten tears. Symptoms include night vision difficulties and diminishing central and peripheral vision. Helicoid peripapillary chorioretinal degeneration is a autosomal dominantly inherited chorioretinal degeneration disease, presenting at birth or infancy, characterized by progressive bilateral retinal and choroidal atrophy, appearing as lesions on the optic nerve and peripheral ocular fundus and leading to blind spots and central vision loss. Congenital anterior polar cataracts are sometimes associated with this disease. Pigmented paravenous retinochoroidal atrophy is characterized by perivenous aggregations of pigment clumps associated with peripapillary and radial zones of retinochoroidal atrophy that are distributed along the retinal veins. Patients with this disorder may be asymptomatic or may have blurred vision.

Retinitis pigmentosa or retinitis is inflammation of the retina of the eye. Retinitis pigmentosa encompasses a group of genetic disorders that involve a breakdown and loss of cells in the retina. As these cells breakdown and die, patients experience progressive vision loss. The most common feature of all forms of retinitis pigmentosa is a gradual breakdown of rods and cones. Most forms of RP first cause the breakdown of rod cells. These forms of retinitis pigmentosa, sometimes called rod-cone dystrophy, usually begin with night blindness. CMV (cytomegalovirus) retinitis develops from a viral infection of the retina. Common symptoms of retinitis pigmentosa include difficulty seeing at night and a loss of side (peripheral) vision through progressive degeneration of the retina. As retinitis pigmentosa progresses, the field of vision narrows, a condition known as “tunnel vision,” until only central vision (the ability to see straight ahead) remains.

Usher syndrome is a genetic disorder, inherited as an autosomal recessive trait, characterized by sensorineural hearing loss or deafness and progressive vision loss due to retinitis pigmentosa.

Retinoblastoma is a type of cancer from genetic mutations that forms in the retina (the light-sensitive tissue at the back of the eye). There are two forms, namely, heritable and non-heritable. It usually develops before the age of 5. The most common first sign of retinoblastoma is a visible whiteness in the pupil called “cat's eye reflex” or leukocoria. Other signs and symptoms of retinoblastoma may include crossed eyes or eyes that do not point in the same direction (strabismus), which can cause squinting; a change in the color of the colored part of the eye (iris); redness, soreness, or swelling of the eyelids; and blindness or poor vision in the affected eye or eyes.

Reticular pseudodrusen are small, gray deposits located above the retinal pigment epithelium. In recent years, reticular pseudodrusen have been recognized as a risk factor for the development of late-stage age-related macular degeneration.

Eye floaters are specks or spots that become evident in the field of vision. They can also look like cobwebs. Floaters can be caused by age-related changes to the vitreous humor, i.e., typically shrinkage which causes microscopic collagenous fibers within the vitreous to clump and cast tiny shadows on the retina. Floaters can also be associated with retinal and posterior vitreous detachments.

Eye flashes can occur when the vitreous humor shrinks and pulls on the retina. They can appear as pin pricks or spots of light, shooting stars or can appear as jagged or wavy streaks of light. Other conditions associated with eye flashes are migraines and detached or torn retinas.

Keratoconus is an eye disorder that affects the structure of the cornea. In keratoconus, the shape of the cornea slowly changes shape from round to a cone shape. It also gets thinner and the eye bulges out. In most people, these changes are progressive. In its earliest stages, keratoconus causes slight blurring and distortion of vision and increased sensitivity to glare and light. These symptoms usually appear in the late teens or late 20s. Keratoconus may progress for 10-20 years and then slow in its progression. As keratoconus progresses, the cornea bulges more and vision can become more distorted. In some cases, the cornea will swell and cause a sudden and significant decrease in vision. The swelling occurs when the strain of the cornea's protruding cone-like shape causes one or more small cracks to develop. The swelling may last for weeks or months as the crack heals and is gradually replaced by scar tissue.

Ocular hypertension occurs when the pressure inside the eye (intraocular pressure) is higher than normal. This may be defined as a pressure greater than 21 mm Hg in one or both eyes. In ocular hypertension, the eye does not drain fluid properly. This causes eye pressure to build up. Higher than normal eye pressure can cause glaucoma. However, ocular hypertension is differentiated from glaucoma. With ocular hypertension, the optic nerve looks normal and there are no signs of vision loss. If high pressure causes damage to the optic nerve, it may lead to glaucoma.

Glaucoma is a group of diseases that damage the eye's optic nerve and can result in vision loss and blindness. Eye pressure is a major risk factor for optic nerve damage. Open-angle glaucoma, is the most common form of the disease. Fluid flows continuously in and out of a chamber in front of the eye called the anterior chamber and nourishes nearby tissues. The fluid leaves the chamber at the open angle where the cornea and iris meet. When the fluid reaches the angle, it flows through a spongy meshwork and leaves the eye. In open-angle glaucoma, even though the drainage angle is “open”, the fluid passes too slowly through the meshwork drain. Since the fluid builds up, the pressure inside the eye rises to a level that may damage the optic nerve. When the optic nerve is damaged from increased pressure, open-angle glaucoma- and vision loss may result.

Presbyopia is a common type of vision disorder that occurs with age. It results in the inability to focus up close, a problem associated with refraction in the eye. In presbyopia, the eye is not able to focus light directly on to the retina due to the hardening of the natural lens. Aging also affects muscle fibers around the lens making it harder for the eye to focus on up close objects. The ineffective lens causes light to focus behind the retina, causing poor vision for objects that are up close.

Dry eye occurs when the quantity and/or quality of tears fails to keep the surface of the eye adequately lubricated. The risk of developing dry eye increases with advancing age. Dry eye causes a scratchy sensation or the feeling that something is in the eye. Other symptoms include stinging or burning, episodes of excess tearing that follow periods of dryness, discharge, pain, and redness in the eye. Dry eye can occur when basal tear production decreases, tear evaporation increases, or tear composition is imbalanced. Dry eye can be caused by: medications including antihistamines, decongestants, antidepressants, birth control pills, hormone replacement therapy to relieve symptoms of menopause, and medications for anxiety, Parkinson's disease, and high blood pressure; rosacea (an inflammatory skin disease) and blepharitis (an inflammatory eyelid disease); autoimmune disorders such as Sjögren's syndrome, lupus, scleroderma, and rheumatoid arthritis and other disorders such as diabetes, thyroid disorders, and Vitamin A deficiency; Seasonal allergies; and windy, smoky, or dry environments can increase tear evaporation.

Bietti's Crystalling Dystrophy (BCD) is an inherited eye disease. Symptoms of BCD include: crystals in the cornea; yellow, shiny deposits on the retina; and progressive atrophy of the retina, choriocapillaries and choroid. This tends to lead to progressive night blindness and visual field constriction.

Behçet's disease is an autoimmune disease that causes inflammation in blood vessels. It causes swelling in some parts of the eye. Inflammatory eye disease can develop early in the disease course and lead to permanent vision loss. Ocular involvement can be in the form of posterior uveitis, anterior uveitis, or retinal vasculitis. Anterior uveitis presents with painful eyes, conjuctival redness, hypopyon, and decreased visual acuity, while posterior uveitis presents with painless decreased visual acuity and visual field floaters. A rare form of ocular involvement in this syndrome is retinal vasculitis which presents with painless decrease of vision with the possibility of floaters or visual field defects.

Achromatopsia 2 is a condition that affects the color vision. Most people have complete achromatopsia which is characterized by a total absence of color vision (only able to see black, white and shades of gray). Rarely, affected people may have incomplete achromatopsia which is associated with some color discrimination. Other common signs and symptoms include reduced visual acuity, involuntary back-and-forth eye movements, increased sensitivity to light (photophobia), and hyperopia (farsightedness). Achromatopsia 2 is believed to be caused by changes (mutations) in the CNGA3 gene and is inherited in an autosomal recessive manner.

Acute posterior multifocal placoid pigment epitheliopathy (APMPPE) is an acquired, inflammatory eye condition affecting the retina, retinal pigment epithelium (pigmented layer of the retina), and choroid. It usually affects both eyes and is characterized by multiple, yellow-white lesions in the back of the eye.

Acute zonal occult outer retinopathy is a rare condition that affects the eyes. People with this condition may experience a sudden onset of photopsia (the presence of perceived flashes of light) and an area of partial vision loss (a blindspot). Other symptoms may include “whitening of vision” or blurred vision.

Ocular albinism with late-onset sensorineural deafness (OASD), is a rare, X-linked inherited type of ocular albinism, characterized by severe visual impairment, translucent pale-blue iridies, a reduction in the retinal pigment and moderately severe deafness by middle age.

Alström syndrome is a rare genetic disorder that affects many body systems. Symptoms develop gradually, beginning in infancy, and can be variable. In childhood, the disorder is generally characterized by vision and hearing abnormalities, childhood obesity, and heart disease (cardiomyopathy). Vision abnormalities, include cone-rod dystrophy and cataracts.

Amyloid corneal dystrophy, aka gelatinous drop-like corneal dystrophy is a form of superficial corneal dystrophy characterized by multiple prominent milky-white gelatinous nodules beneath the corneal epithelium, photophobia and marked visual impairment.

Anterior ischemic optic neuropathy is an eye disorder characterized by infarction of the optic disk leading to vision loss. It can be nonarteritic (nonarteritic anterior ischemic optic neuropathy) or arteritic, the latter being associated with giant cell arteritis (GCA; often termed temporal arteritis).

Axenfeld-Rieger syndrome is a group of disorders that mainly affects the development of the eye. Common eye symptoms include cornea defects and iris defects. People with this syndrome may have an off-center pupil (corectopia) or extra holes in the eyes that can look like multiple pupils (polycoria). People with this disorder typically have cornea defects. They may have a cloudy cornea or posterior embryotoxin, an opaque ring around the outer edge of the cornea. People with this disorder can also have issues with their iris such as iris stands, which is connective tissue that connects the iris with the lens.

Bardet-Biedl syndrome is an inherited condition that affects many parts of the body. People with this syndrome have progressive visual impairment due to cone-rod dystrophy. Progressive vision loss due to deterioration of the retina. This usually begins in mid-childhood with problems with night vision, followed by the development of blind spots in peripheral vision. Blind spots become bigger with time and eventually merge to produce tunnel vision. Most individuals also develop blurred central vision and become legally blind by adolescence or early adulthood (over 90% of cases).

Behr syndrome is a disorder characterized by early-onset optic atrophy along with neurological features, including ataxia, spasticity, and intellectual disability. People with Behr syndrome typically have visual disturbances (e.g. optic atrophy, nystagmus, scotoma, and bilateral retrobulbar neuritis).

Bietti crystalline corneoretinal dystrophy is an inherited eye disease. Symptoms include crystals in the cornea (the clear covering of the eye); yellow, shiny deposits on the retina; and progressive atrophy of the retina, choriocapillaries and choroid (the back layers of the eye). This tends to lead to progressive night blindness and loss of visual acuity.

Birdshot chorioretinopathy is an eye condition in which painless, light-colored spots develop on the retina. These spots are scattered in a “birdshot” pattern. The effects of this condition on vision are quite variable; some individuals' vision is only mildly affected, whereas others experience a significant decline in vision, the appearance of floaters, night blindness, and other vision problems. Symptoms typically begin around middle age.

Blue cone monochromatism is an inherited vision disorder. In this condition, the light sensitive cells in the eye used for color vision (cones) are affected. There are three types of cones that respond to one of three colors: red, green, and blue. When people have blue cone monochromatism, both the red and green cones do not function properly, while the blue cones work normally.

Coats disease is an eye disorder characterized by abnormal development of the blood vessels in the retina (retinal telangiectasia). Most people begin displaying symptoms in childhood. Early signs and symptoms vary but may include vision loss, “crossed eyes” (strabismus), and a white mass in the pupil behind the lens of the eye (leukocoria). Over time, Coats disease may also lead to retinal detachment, glaucoma, and clouding of the lens of the eye (cataracts).

Iridocorneal endothelial syndrome describes a group of eye diseases that are characterized by three main features: 1) visible changes in the iris (the colored part of the eye that regulates the amount of light entering the eye), 2) swelling of the cornea, and 3) development of glaucoma.

Corneal dystrophy, Avellino type is an inherited condition that affects the stromal or central layer of the cornea. It results in the development of small particles or granules (like breadcrumbs) on the cornea (known as granular corneal dystrophy) and the development of lesions that resemble cracked glass (known as lattice corneal dystrophy). These eye lesions usually develop on the stromal layer before age 20. As affected individuals age, the lesions may become larger, more prominent, and involve the entire stromal layer.

Schnyder corneal dystrophy is a rare form of stromal corneal dystrophy characterized by corneal clouding or crystals within the corneal stroma, and a progressive decrease in visual acuity.

Thiel-Behnke corneal dystrophy is a rare form of superficial corneal dystrophy characterized by sub-epithelial honeycomb-shaped corneal opacities in the superficial cornea, and progressive visual impairment.

Eales disease is a rare vision disorder that appears as an inflammation and white haze around the outercoat of the veins in the retina. This condition is most common among young males and normally affects both eyes. In most cases, vision becomes suddenly blurred because the vitreous seeps out.

Epithelial basement membrane corneal dystrophy is a condition where the epithelium of the cornea (the outermost region of the cornea) loses its normal clarity due to a buildup of cloudy material. This dystrophy occurs when the epithelium's basement membrane develops abnormally, causing the epithelial cells to not properly adhere to it.

Fish-eye disease is a rare ocular condition. People with this condition generally develop corneal clouding beginning in adolescence or early adulthood. Overtime, the condition gradually worsens and can lead to significant vision loss.

Fuchs endothelial corneal dystrophy is an eye disorder that affects the thin layer of cells that line the back part of the cornea (endothelium). It is manifest when these cells slowly start to die off. These cells help pump excess fluid out of the cornea. As more and more cells are lost, fluid begins to build up in the cornea, causing swelling and a cloudy cornea.

Goldmann-Favre syndrome, also known as the severe form of enhanced S-cone syndrome, is an inherited eye disease that affects the retina. Within the retina are “red,” “blue,” and “green” cones for visualizing color; and rods which allows sight in dim light. People with Goldmann-Favre syndrome are born with an overabundance of blue cones, a reduced number of red and green cones, and few, if any, functional rods.

Late-onset retinal degeneration is an inherited retinal dystrophy characterized by delayed dark adaptation and nyctalopia and drusen deposits presenting in adulthood, followed by cone and rod degeneration that presents in the sixth decade of life, which leads to central vision loss.

Leber congenital amaurosis is an eye disorder that primarily affects the retina. People with this condition typically have severe visual impairment beginning in infancy. Other features include photophobia, involuntary movements of the eyes (nystagmus), and extreme farsightedness. The pupils also do not react normally to light. Additionally, the cornea may be cone-shaped and abnormally thin (keratoconus).

Peters anomaly is a disorder of the eye which involves thinning and clouding of the cornea and attachment of the iris to the cornea, which causes blurred vision. It may also be associated with clouding of the lens of the eye (cataracts) or other lens abnormalities.

Punctate inner choroidopathy is an inflammatory disorder that primarily affects the choroid of the eye and occurs predominantly in young, nearsighted (myopic) women. Signs and symptoms may include scotomata, blurred vision, photopsias, floaters, photophobia, distorted vision (metamorphopsia), and/or loss of peripheral vision.

Senior Loken syndrome (SLS) is a rare syndrome that mainly affects the kidneys and eyes. SLS affects the eyes by causing varying degrees of retinal dystrophy, which is inherited progressive wasting of the retina. Some children with SLS have a severe type of retinal dystrophy at birth called Leber congenital amaurosis (LCA). Symptoms of LCA include severe farsightedness, light sensitivity (photophobia), and nystagmus.

Snowflake vitreoretinal degeneration is characterised by the presence of small granular-like deposits resembling snowflakes in the retina, fibrillary vitreous degeneration and cataract.

Visual snow syndrome causes a person to see numerous snow-like flickering tiny dots that fill the entire visual field in both eyes. For most people with the syndrome, the visual snow is always present and occurs in both eyes. The visual snow may worsen at times when the brain and eyes are “tired”, such as after looking at a computer screen for a long time or during times of stress. Other visual symptoms that can be associated with visual snow syndrome include sensitivity to light (photophobia), continuing to see an image after it is no longer in the field of vision (palinopsia), impaired night vision (nyctalopia), and seeing images from within the eye itself (entoptic phenomena), such as seeing small floating objects or flashes of light.

Wagner syndrome is a hereditary eye disorder that leads to progressive vision loss. It is characterized by changes to the vitreous, which it becomes thin and watery and appears empty. The first signs and symptoms usually appear in childhood, but onset may be as early as age 2. Signs and symptoms may include: thinning of the light-sensitive tissue that lines the back of the eye (retinal detachment), abnormalities of the blood vessels within the retina (the choroid), and degeneration of the retina and choroid.

In embodiments, provided herein are methods of treating an eye disorder including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the eye disorder. Symptoms of the above-listed eye disorders may include, but are not limited to, vision loss, drusen, pigment changes in the retina, abnormal blood vessel growth, leaky blood vessels, macular swelling, corneal swelling, corneal thinning, accumulation of a fatty yellow pigment (lipofuscin), night blindness, distorted vision, blurry vision, rod damage, cone damage, uvea inflammation, eye redness, pain, sensitivity to light (photophobia), floaters, eye flashes, nodules, orbital inflammation, lacrimal gland enlargement, decreased visual acuity, decrease in contrast sensitivity, blind spots, loss of color perception, loss of peripheral vision, fluid build-up in the macula, retinal scarring, double vision, pigment clumps, tunnel vision, thin cornea, spotting, leukocoria, lesions, crystals, nystagmus, and any other symptoms associated with the above-listed disorders. It should be understood that each of the above-listed eye disorders may have one or more of the above-listed symptoms.

Methods of treating Stargardt disease are provided and, in embodiments, include administering to a subject in need thereof an effective amount of (1 S,3 S)-3-amino-4-(difluoromethylidene)cyclopentane-1-carboxylic acid or a pharmaceutically acceptable salt thereof. In embodiments, methods of treating Stargardt disease include administering (1 S,3 S)-3-amino-4-(difluoromethylidene)cyclopentane-1-carboxylic acid or a pharmaceutically acceptable salt thereof to a subject in need thereof to provide improvement in one or more symptoms of the Stargardt disease in the subject. In embodiments, methods of treating Stargardt disease include administering (1 S,3 S)-3-amino-4-(difluoromethylidene)cyclopentane-1-carboxylic acid or a pharmaceutically acceptable salt thereof to a subject in need thereof to provide improvement in Stargardt disease symptoms of the subject the next day after administration. In embodiments, methods of treating Stargardt disease include administering to a subject in need thereof an effective amount of (S)-3-amino-4-(difluoromethylenyl)cyclopent-1-ene-1-carboxylic acid or a pharmaceutically acceptable salt thereof. In embodiments, methods of treating Stargardt disease include administering (S)-3-amino-4-(difluoromethylenyl)cyclopent-1-ene-1-carboxylic acid or a pharmaceutically acceptable salt thereof to a subject in need thereof to provide improvement in one or more symptoms of the Stargardt disease. In embodiments, methods of treating Stargardt disease include administering (S)-3-amino-4-(difluoromethylenyl)cyclopent-1-ene-1-carboxylic acid or a pharmaceutically acceptable salt thereof to a subject in need thereof to provide improvement in Stargardt disease symptoms of the subject the next day after administration. In embodiments, methods of treating Stargardt disease include administering (1S,3S)-3-amino-4-(difluoromethylidene)cyclopentane-1-carboxylic acid or a pharmaceutically acceptable salt thereof, in combination with (S)-3-amino-4-(difluoromethylenyl)cyclopent-1-ene-1-carboxylic acid or a pharmaceutically acceptable salt thereof, to a subject in need thereof.

In embodiments, provided herein are methods of treating Stargardt disease including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or combinations of the foregoing, wherein the composition provides improvement in at least one symptom of Stargardt disease. Symptoms of Stargardt disease may include, but are not limited to, accumulation of a fatty yellow pigment (lipofuscin), vision loss, night blindness, lack of visual acuity, and loss of color perception.

In embodiments, provided herein are methods of treating juvenile macular degeneration including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or combinations of the foregoing, wherein the composition provides improvement in at least one symptom of the juvenile macular degeneration.

In embodiments, provided herein are methods of treating age related macular degeneration including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the age related macular degeneration.

In embodiments, provided herein are methods of treating Best disease including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the Best disease.

In embodiments, provided herein are methods of treating juvenile retinoschisis including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or combinations of the foregoing, wherein the composition provides improvement in at least one symptom of the juvenile retinoschisis.

In embodiments, provided herein are methods of treating Doyne honeycomb retinal dystrophy including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the Doyne honeycomb retinal dystrophy.

In embodiments, provided herein are methods of treating uveitis including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the uveitis.

In embodiments, provided herein are methods of treating scleritis including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the scleritis.

In embodiments, provided herein are methods of treating ocular sarcoidosis including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the ocular sarcoidosis.

In embodiments, provided herein are methods of treating optic neuritis including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the optic neuritis.

In embodiments, provided herein are methods of treating cone-rod dystrophy including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the cone-rod dystrophy.

In embodiments, provided herein are methods of treating diabetic retinopathy including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the diabetic retinopathy.

In embodiments, provided herein are methods of treating optic nerve degeneration including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the optic nerve degeneration.

In embodiments, provided herein are methods of treating a choroidal dystrophy including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the choroidal dystrophy.

In embodiments, provided herein are methods of treating retinitis pigmentosa including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the retinitis pigmentosa.

In embodiments, provided herein are methods of treating keratoconus including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the keratoconus.

In embodiments, provided herein are methods of treating macular edema including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the macular edema.

In embodiments, provided herein are methods of treating Bietti crystalline corneoretinal dystrophy including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the Bietti crystalline corneoretinal dystrophy.

In embodiments, provided herein are methods of treating birdshot chorioretinopathy including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the birdshot chorioretinopathy.

In embodiments, provided herein are methods of treating Coats disease including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the Coats disease.

In embodiments, provided herein are methods of treating punctate inner choroidopathy including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the punctate inner choroidopathy.

In embodiments, provided herein are methods of treating Wagner syndrome including administering to a patient in need thereof a pharmaceutical composition including (1S,3S)-3-amino-4-(difluoromethylidene) cyclopentane-1-carboxylic acid or a pharmaceutically acceptable salt thereof, or (S)-3-amino-4-(difluoromethylenyl)cyclopent-1-ene-1-carboxylic acid or a pharmaceutically acceptable salt thereof, or a combination of the foregoing, wherein the composition provides improvement in at least one symptom of the Wagner syndrome.

As used herein, the terms “effective amount” or “therapeutically effective amount” refer to an amount of a compound, material, composition, medicament, or other material that is effective to achieve a particular pharmacological and/or physiologic effect in connection with symptoms of eye disorders herein. Likewise, the terms “effective amount” or “therapeutically effective amount” refer to an amount of a compound, material, composition, medicament, or other material that is effective to achieve a particular pharmacological and/or physiologic effect in connection with an eye disorder such as Stargardt disease.

Accordingly, an effective amount of (1 S,3 S)-3-amino-4-(difluoromethylidene)cyclopentane-1-carboxylic acid or a pharmaceutically acceptable salt thereof is used to treat a subject having Stargardt disease. An effective amount of (1 S, 3 S)-3-amino-4-(difluoromethylidene)cyclopentane-1-carboxylic acid or a pharmaceutically acceptable salt thereof is used to treat a subject having juvenile macular degeneration. An effective amount of (1S,3S)-3-amino-4-(difluoromethylidene)cyclopentane-1-carboxylic acid or a pharmaceutically acceptable salt thereof is used to treat a subject having Best disease. Accordingly, an effective amount of (1S,3 S)-3-amino-4-(difluoromethylidene)cyclopentane-1-carboxylic acid or a pharmaceutically acceptable salt thereof is used to treat a subject having juvenile retinoschisis. An effective amount of (1S,3S)-3-amino-4-(difluoromethylidene)cyclopentane-1-carboxylic acid or a pharmaceutically acceptable salt thereof is used to treat a subject having Doyne honeycomb retinal dystrophy. An effective amount of (1 S,3 S)-3-amino-4-(difluoromethylidene)cyclopentane-1-carboxylic acid or a pharmaceutically acceptable salt thereof is used to treat a subject having uveitis. Accordingly, an effective amount of (1 S,3 S)-3-amino-4-(difluoromethylidene)cyclopentane-1-carboxylic acid or a pharmaceutically acceptable salt thereof is used to treat a subject having scleritis. An effective amount of (1 S,3 S)-3-amino-4-(difluoromethylidene)cyclopentane-1-carboxylic acid or a pharmaceutically acceptable salt thereof is used to treat a subject having ocular sarcoidosis. An effective amount of (1 S,3 S)-3-amino-4-(difluoromethylidene)cyclopentane-1-carboxylic acid or a pharmaceutically acceptable salt thereof is used to treat a subject having optic neuritis. Accordingly, an effective amount of (1S,3 S)-3-amino-4-(difluoromethylidene)cyclopentane-1-carboxylic acid or a pharmaceutically acceptable salt thereof is used to treat a subject having cone-rod dystrophy. An effective amount of (1 S,3 S)-3-amino-4-(difluoromethylidene)cyclopentane-1-carboxylic acid or a pharmaceutically acceptable salt thereof is used to treat a subject having diabetic retinopathy. An effective amount of (1S,3S)-3-amino-4-(difluoromethylidene)cyclopentane-1-carboxylic acid or a pharmaceutically acceptable salt thereof is used to treat a subject having optic nerve degeneration. Accordingly, an effective amount of (1 S,3 S)-3-amino-4-(difluoromethylidene)cyclopentane-1-carboxylic acid or a pharmaceutically acceptable salt thereof is used to treat a subject having choroidal dystrophy. An effective amount of (1 S,3 S)-3-amino-4-(difluoromethylidene)cyclopentane-1-carboxylic acid or a pharmaceutically acceptable salt thereof is used to treat a subject having retinitis pigmentosa. An effective amount of (1 S, 3 S)-3-amino-4-(difluoromethylidene)cyclopentane-1-carboxylic acid or a pharmaceutically acceptable salt thereof is used to treat a subject having keratoconus. An effective amount of (1 S,3 S)-3-amino-4-(difluoromethylidene)cyclopentane-1-carboxylic acid or a pharmaceutically acceptable salt thereof is used to treat a subject having Bietti crystalline corneoretinal dystrophy. An effective amount of (1 S,3 S)-3-amino-4-(difluoromethylidene)cyclopentane-1-carboxylic acid or a pharmaceutically acceptable salt thereof is used to treat a subject having birdshot chorioretinopathy. Accordingly, an effective amount of (1 S,3 S)-3-amino-4-(difluoromethylidene)cyclopentane-1-carboxylic acid or a pharmaceutically acceptable salt thereof is used to treat a subject having Coats disease. An effective amount of (1 S,3 S)-3-amino-4-(difluoromethylidene)cyclopentane-1-carboxylic acid or a pharmaceutically acceptable salt thereof is used to treat a subject having punctate inner choroidopathy. An effective amount of (1S,3S)-3-amino-4-(difluoromethylidene)cyclopentane-1-carboxylic acid or a pharmaceutically acceptable salt thereof is used to treat a subject having Wagner syndrome.

Accordingly, an effective amount of (S)-3-amino-4-(difluoromethylenyl)cyclopent-1-ene-1-carboxylic acid or a pharmaceutically acceptable salt thereof is used to treat a subject having Stargardt disease. An effective amount of (S)-3-amino-4-(difluoromethylenyl)cyclopent-1-ene-1-carboxylic acid or a pharmaceutically acceptable salt thereof is used to treat a subject having juvenile macular degeneration. An effective amount of (S)-3-amino-4-(difluoromethylenyl)cyclopent-1-ene-1-carboxylic acid or a pharmaceutically acceptable salt thereof is used to treat a subject having Best disease. Accordingly, an effective amount of (S)-3-amino-4-(difluoromethylenyl)cyclopent-1-ene-1-carboxylic acid or a pharmaceutically acceptable salt thereof is used to treat a subject having juvenile retinoschisis. An effective amount of (S)-3-amino-4-(difluoromethylenyl)cyclopent-1-ene-1-carboxylic acid or a pharmaceutically acceptable salt thereof is used to treat a subject having Doyne honeycomb retinal dystrophy. An effective amount (S)-3-amino-4-(difluoromethylenyl)cyclopent-1-ene-1-carboxylic acid or a pharmaceutically acceptable salt thereof is used to treat a subject having uveitis. Accordingly, an effective amount of (S)-3-amino-4-(difluoromethylenyl)cyclopent-1-ene-1-carboxylic acid or a pharmaceutically acceptable salt thereof is used to treat a subject having scleritis. An effective amount of (S)-3-amino-4-(difluoromethylenyl)cyclopent-1-ene-1-carboxylic acid or a pharmaceutically acceptable salt thereof is used to treat a subject having ocular sarcoidosis. An effective amount of (S)-3-amino-4-(difluoromethylenyl)cyclopent-1-ene-1-carboxylic acid or a pharmaceutically acceptable salt thereof is used to treat a subject having optic neuritis. Accordingly, an effective amount of (S)-3-amino-4-(difluoromethylenyl)cyclopent-1-ene-1-carboxylic acid or a pharmaceutically acceptable salt thereof is used to treat a subject having cone-rod dystrophy. An effective amount of (S)-3-amino-4-(difluoromethylenyl)cyclopent-1-ene-1-carboxylic acid or a pharmaceutically acceptable salt thereof is used to treat a subject having diabetic retinopathy. An effective amount of (S)-3-amino-4-(difluoromethylenyl)cyclopent-1-ene-1-carboxylic acid or a pharmaceutically acceptable salt thereof is used to treat a subject having optic nerve degeneration. Accordingly, an effective amount of (S)-3-amino-4-(difluoromethylenyl)cyclopent-1-ene-1-carboxylic acid or a pharmaceutically acceptable salt thereof is used to treat a subject having choroidal dystrophy. An effective amount of (S)-3-amino-4-(difluoromethylenyl)cyclopent-1-ene-1-carboxylic acid or a pharmaceutically acceptable salt thereof is used to treat a subject having retinitis pigmentosa. An effective amount of (1 S,3 S)-3-amino-4-(difluoromethylidene)cyclopentane-1-carboxylic acid or a pharmaceutically acceptable salt thereof is used to treat a subject having keratoconus. An effective amount of (S)-3-amino-4-(difluoromethylenyl)cyclopent-1-ene-1-carboxylic acid or a pharmaceutically acceptable salt thereof is used to treat a subject having Bietti crystalline corneoretinal dystrophy. An effective amount of (S)-3-amino-4-(difluoromethylenyl)cyclopent-1-ene-1-carboxylic acid or a pharmaceutically acceptable salt thereof is used to treat a subject having birdshot chorioretinopathy. Accordingly, an effective amount of (S)-3-amino-4-(difluoromethylenyl)cyclopent-1-ene-1-carboxylic acid or a pharmaceutically acceptable salt thereof is used to treat a subject having Coats disease. An effective amount of (S)-3-amino-4-(difluoromethylenyl)cyclopent-1-ene-1-carboxylic acid or a pharmaceutically acceptable salt thereof is used to treat a subject having punctate inner choroidopathy. An effective amount of (S)-3-amino-4-(difluoromethylenyl)cyclopent-1-ene-1-carboxylic acid or a pharmaceutically acceptable salt thereof is used to treat a subject having Wagner syndrome.

The subject may be an animal, e.g., mammal, e.g., human, etc. As used herein, the terms “treat”, “treatment” or “treating” encompass any manner in which the symptoms or pathology of a condition, disorder or disease associated with an eye disorder such as Stargardt disease are ameliorated or otherwise beneficially altered. Indeed, “treat”, “treatment” or “treating” encompass any manner in which the symptoms or pathology of a condition, disorder or disease associated with Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, or Wagner syndrome, are ameliorated or otherwise beneficially altered. In embodiments, “treat”, “treatment” or “treating” can refer to inhibiting a disorder, disease or condition, e.g., arresting or reducing its development or at least one clinical or subclinical symptom thereof. In embodiments, “treat”, “treatment” or “treating” can refer to relieving the disease or condition, e.g., causing regression of the disorder, disease or condition or at least one of its clinical or subclinical symptoms. The benefit to a subject being treated may be statistically significant, mathematically significant, or at least perceptible to the subject and/or the physician.

The effective amount can vary according to a variety of factors such as subject-dependent variables (e.g., age, immune system, health, etc.), the disease or disorder being treated, as well as the route of administration and the pharmacokinetics of the agent being administered.

Many pharmaceutical products are administered as a fixed dose, at regular intervals, to achieve therapeutic efficacy. Duration of action is typically reflected by a drug's plasma half-life. Since efficacy is often dependent on sufficient exposure within a target area, administration of drugs with a short half-life may require frequent maintenance dosing. The plasma elimination half-life of (1S,3 S)-3-amino-4-(difluoromethylidene)cyclopentane-1-carboxylic acid is between about 4 to 6 hours. Cₘₐₓ increases in a dose proportional manner over a range of 5 mg-500 mg; whereas there is a greater than proportional increase in AUCs in the dose range. (S)-3-amino-4-(difluoromethylenyl)cyclopent-1-ene-1-carboxylic acid is between 9 and 10 times more potent as an inactivator of GABA-AT than (1S,3S)-3-amino-4-(difluoromethylidene)cyclopentane-1-carboxylic acid and may exhibit similar pharmacokinetics.

In embodiments, (1 S,3 S)-3-amino-4-(difluoromethylidene)cyclopentane-1-carboxylic acid may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, pantothenic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, hydroiodic, sulfuric, sulfamic, phosphoric or nitric acid addition salts may be used.

In embodiments, (S)-3-amino-4-(difluoromethylenyl)cyclopent-1-ene-1-carboxylic acid may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, pantothenic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, hydroiodic, sulfuric, sulfamic, phosphoric or nitric acid addition salts may be used.

In embodiments, methods include treating Stargardt disease by administering to a subject in need thereof about 0.1 mg to about 1500 mg of (1S,3S)-3-amino-4-difluoromethylenyl-1-cyclopentanoic acid or a pharmaceutically acceptable salt thereof, e.g., a hydrochloride salt thereof. In embodiments, methods include treating Stargardt disease by administering to a subject in need thereof about 0.5 mg to about 1000 mg of (1S,3S)-3-amino-4-difluoromethylenyl-1-cyclopentanoic acid or a pharmaceutically acceptable salt thereof, e.g., a hydrochloride salt thereof. In embodiments, the amount of (1S,3S)-3-amino-4-difluoromethylenyl-1-cyclopentanoic acid or a pharmaceutically acceptable salt thereof, e.g., a hydrochloride salt thereof, can be between 0.1 and 1500 mg/day, or 0.01 mg/kg/day to 15 mg/kg/day, for treatment of Stargardt disease. In embodiments, the amount of (1S,3S)-3-amino-4-difluoromethylenyl-1-cyclopentanoic acid or a pharmaceutically acceptable salt thereof, e.g., a hydrochloride salt thereof, can be between 0.1 and 1000 mg/day for treatment Stargardt disease.

In embodiments, methods include treating an eye disorder by administering to a subject in need thereof about 0.1 mg to about 1500 mg of (1S,3S)-3-amino-4-difluoromethylenyl-1-cyclopentanoic acid or a pharmaceutically acceptable salt thereof, e.g., a hydrochloride salt thereof. In embodiments, methods include treating an eye disorder by administering to a subject in need thereof about 0.5 mg to about 1000 mg of (1S,3S)-3-amino-4-difluoromethylenyl-1-cyclopentanoic acid or a pharmaceutically acceptable salt thereof, e.g., a hydrochloride salt thereof. In embodiments, the amount of (1S,3S)-3-amino-4-difluoromethylenyl-1-cyclopentanoic acid or a pharmaceutically acceptable salt thereof, e.g., a hydrochloride salt thereof, can be between 0.1 and 1500 mg/day, or 0.01 mg/kg/day to 15 mg/kg/day, for treatment of an eye disorder. In embodiments, the amount of (1S,3S)-3-amino-4-difluoromethylenyl-1-cyclopentanoic acid or a pharmaceutically acceptable salt thereof, e.g., a hydrochloride salt thereof, can be between 0.1 and 1000 mg/day for treatment an eye disorder. In embodiments, the maximum amount of (1S,3S)-3-amino-4-difluoromethylenyl-1-cyclopentanoic acid or a pharmaceutically acceptable salt thereof, e.g., a hydrochloride salt thereof, can be 500 mg/day for treatment an eye disorder. Such eye disorders are Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy (APMPPE), acute zonal occult outer retinopathy (AZOOR), adult-onset vitelliform macular dystrophy (AVMD), ocular albinism with late-onset sensorineural deafness (OASD), Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome.

For example, the daily dosage of (1S,3S)-3-amino-4-difluoromethylenyl-1-cyclopentanoic acid or a pharmaceutically acceptable salt thereof can be, e.g., in the range of about 0.1 to 1500 mg, 0.1 to 1250 mg, 0.1 to 1000 mg, 0.1 to 750 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 1 to 1500 mg, 1 to 1000 mg, 1 to 500 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 1500 mg, 5 to 1000 mg, 5 to 500 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 1500 mg, 10 to 1000 mg, 10 to 500 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 1500 mg, 15 to 1000 mg, 15 to 500 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 1500 mg, 20 to 1000 mg, 20 to 500 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 1500 mg, 25 to 1000 mg, 25 to 500 mg, 25 to 300 mg, 25 to 250 mg, 25 to 200 mg, 25 to 175 mg, 25 to 150 mg, 25 to 125 mg, 25 to 100 mg, 25 to 75 mg, 25 to 50 mg, 25 to 30 mg, 30 to 1500 mg, 30 to 1000 mg, 30 to 500 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 35 to 1500 mg, 35 to 1000 mg, 35 to 500 mg, 35 to 300 mg, 35 to 250 mg, 35 to 200 mg, 35 to 175 mg, 35 to 150 mg, 35 to 125 mg, 35 to 100 mg, 35 to 75 mg, 35 to 50 mg, 40 to 1500 mg, 40 to 1000 mg, 40 to 500 mg, 40 to 300 mg, 40 to 250 mg, 40 to 200 mg, 40 to 175 mg, 40 to 150 mg, 40 to 125 mg, 40 to 100 mg, 40 to 75 mg, 40 to 50 mg, 50 to 1500 mg, 50 to 1000 mg, 50 to 500 mg, 50 to 300 mg, 50 to 250 mg, 50 to 200 mg, 50 to 175 mg, 50 to 150 mg, 50 to 125 mg, 50 to 100 mg, 50 to 75 mg, 75 to 1500 mg, 75 to 1000 mg, 75 to 500 mg, 75 to 300 mg, 75 to 250 mg, 75 to 200 mg, 75 to 175 mg, 75 to 150 mg, 75 to 125 mg, 75 to 100 mg, 100 to 1500 mg, 100 to 1000 mg, 100 to 500 mg, 100 to 300 mg, 100 to 250 mg, 100 to 200 mg, 100 to 175 mg, 100 to 150 mg, 100 to 125 mg, 125 to 1500 mg, 125 to 1000 mg, 125 to 500 mg, 125 to 300 mg, 125 to 250 mg, 125 to 200 mg, 125 to 175 mg, 125 to 150 mg, 150 to 1500 mg, 150 to 1000 mg, 150 to 500 mg, 150 to 300 mg, 150 to 250 mg, 150 to 200 mg, 150 to 175 mg, 175 to 1500 mg, 175 to 1000 mg, 175 to 500 mg, 175 to 300 mg, 175 to 250 mg, 175 to 200 mg, 200 to 1500 mg, 200 to 1000 mg, 200 to 500 mg, 200 to 300 mg, 200 to 250 mg, 250 to 1500 mg, 250 to 1000 mg, 250 to 500 mg, 250 to 300 mg, 7.5 to 15 mg, 2.5 to 5 mg, 1 to 5 mg, with doses of, e.g., about 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 7.5 mg, 10 mg, 12.5 mg, 15 mg, 17.5 mg, 20 mg, 22.5 mg, 25 mg, 27.5 mg, 30 mg, 35 mg, 40 mg, 45 mg, 50 mg, 55 mg, 60 mg, 65 mg, 70 mg, 75 mg, 100 mg, 125 mg, 150 mg, 175 mg, 200 mg, 225 mg, 250 mg, 275 mg, 300 mg, 400 mg and 500 mg being examples.

In embodiments, pharmaceutical compositions may include (1S,3S)-3-amino-4-difluoromethylenyl-1-cyclopentanoic acid or a pharmaceutically acceptable salt thereof in an amount of, e.g., about 0.01 to 500 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 0.5 to 500 mg, 0.5 to 450 mg, 0.5 to 300 mg, 0.5 to 250 mg, 0.5 to 200 mg, 0.5 to 175 mg, 0.5 to 150 mg, 0.5 to 125 mg, 0.5 to 100 mg, 0.5 to 75 mg, 0.5 to 50 mg, 0.5 to 30 mg, 0.5 to 25 mg, 0.5 to 20 mg, 0.5 to 15 mg, 0.5 to 10 mg, 0.5 to 5 mg, 0.5 to 1 mg, 1 to 500 mg, 1 to 450 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 500 mg, 5 to 450 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 500 mg, 10 to 450 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 500 mg, 15 to 450 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 500 mg, 20 to 450 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 500 mg, 25 to 450 mg, 25 to 300 mg, 25 to 250 mg, 25 to 200 mg, 25 to 175 mg, 25 to 150 mg, 25 to 125 mg, 25 to 100 mg, 25 to 80 mg, 25 to 75 mg, 25 to 50 mg, 25 to 30 mg, 30 to 500 mg, 30 to 450 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 40 to 500 mg, 40 to 450 mg, 40 to 400 mg, 40 to 250 mg, 40 to 200 mg, 40 to 175 mg, 40 to 150 mg, 40 to 125 mg, 40 to 100 mg, 40 to 75 mg, 40 to 50 mg, 50 to 500 mg, 50 to 450 mg, 50 to 300 mg, 50 to 250 mg, 50 to 200 mg, 50 to 175 mg, 50 to 150 mg, 50 to 125 mg, 50 to 100 mg, 50 to 75 mg, 75 to 500 mg, 75 to 450 mg, 75 to 300 mg, 75 to 250 mg, 75 to 200 mg, 75 to 175 mg, 75 to 150 mg, 75 to 125 mg, 75 to 100 mg, 100 to 500 mg, 100 to 450 mg, 100 to 300 mg, 100 to 250 mg, 100 to 200 mg, 100 to 175 mg, 100 to 150 mg, 100 to 125 mg, 125 to 500 mg, 125 to 450 mg, 125 to 300 mg, 125 to 250 mg, 125 to 200 mg, 125 to 175 mg, 125 to 150 mg, 150 to 500 mg, 150 to 450 mg, 150 to 300 mg, 150 to 250 mg, 150 to 200 mg, 200 to 500 mg, 200 to 450 mg, 200 to 300 mg, 200 to 250 mg, 250 to 500 mg, 250 to 450 mg, 250 to 300 mg, 300 to 500 mg, 300 to 450 mg, 300 to 400 mg, 300 to 350 mg, 350 to 500 mg, 350 to 450 mg, 350 to 400 mg, 400 to 500 mg, 400 to 450 mg, with 0.1 mg, 0.25 mg, 0.5 mg, 0.75 mg, 1 mg, 2.5 mg, 5 mg, 7.5 mg, 10 mg, 12.5 mg, 15 mg, 17.5 mg, 20 mg, 22.5 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg 175 mg, 200 mg, 225 mg, 250 mg, 275 mg, 300 mg, 325 mg, 350 mg, 375 mg, 400 mg, 425 mg, 450 mg, 475 mg, and 500 mg being examples.

Typically, dosages for treating Stargardt disease may be administered to a subject once, twice, three or four times daily, every other day, once weekly, or once a month. In embodiments, (1S,3S)-3-amino-4-difluoromethylenyl-1-cyclopentanoic acid or a pharmaceutically acceptable salt thereof is administered to a subject twice a day, (e.g., morning and evening), or three times a day (e.g., at breakfast, lunch, and dinner), at a dose of 1-50 mg/administration. In embodiments, (1S,3S)-3-amino-4-difluoromethylenyl-1-cyclopentanoic acid or a pharmaceutically acceptable salt thereof is administered to a subject 100 mg/per day, 95 mg/per day, 90 mg/per day, 85 mg/per day, 80 mg/per day, 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 5 mg/per day, 4 mg/per day, 3 mg/per day, 2 mg/per day, 1 mg/per day, in one or more doses. In embodiments, an adult dose for Stargardt disease can be about 5 to 80 mg per day and can be increased to 150 mg per day. Dosages can be lower for infants and children than for adults. In embodiments, a pediatric dose for treating Stargardt disease can be about 0.1 to 50 mg per day once or in 2, 3 or 4 divided doses. In embodiments, a pediatric dose for treating Stargardt disease can be 0.2 mg/kg/day to 1.5 mg/kg/day. In embodiments, the subject may be started at a low dose and the dosage is escalated over time.

Typically, dosages for treating one or more of the following eye disorders: macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome, may be administered to a subject once, twice, three or four times daily, every other day, once weekly, or once a month. In embodiments, (1S,3S)-3-amino-4-difluoromethylenyl-1-cyclopentanoic acid or a pharmaceutically acceptable salt thereof is administered to a subject twice a day, (e.g., morning and evening), or three times a day (e.g., at breakfast, lunch, and dinner), at a dose of 1-50 mg/administration. In embodiments, (1S,3S)-3-amino-4-difluoromethylenyl-1-cyclopentanoic acid or a pharmaceutically acceptable salt thereof is administered to a subject 100 mg/per day, 95 mg/per day, 90 mg/per day, 85 mg/per day, 80 mg/per day, 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 5 mg/per day, 4 mg/per day, 3 mg/per day, 2 mg/per day, 1 mg/per day, in one or more doses. In embodiments, an adult dose for the eye disorder can be about 5 to 80 mg per day and can be increased to 150 mg per day. Dosages can be lower for infants and children than for adults. In embodiments, a pediatric dose for treating the eye disorder can be about 0.1 to 50 mg per day once or in 2, 3 or 4 divided doses. In embodiments, a pediatric dose for treating the eye disorder can be 0.75 mg/kg/day to 1.5 mg/kg/day. In embodiments, the subject may be started at a low dose and the dosage is escalated over time.

In embodiments, (1S,3S)-3-amino-4-difluoromethylenyl-1-cyclopentanoic acid or a pharmaceutically acceptable salt thereof is administered via a pharmaceutical composition. Pharmaceutical compositions (also referred to simply as compositions) herein encompass dosage forms. Dosage forms herein encompass unit doses. In embodiments, as discussed below, various dosage forms including conventional formulations and modified release formulations can be administered one or more times daily. Any suitable route of administration may be utilized, e.g., oral, rectal, nasal, ophthalmic, pulmonary, vaginal, sublingual, transdermal, intravenous, intraarterial, intramuscular, intraperitoneal and subcutaneous routes. Suitable dosage forms include tablets, capsules, oral liquids, ophthalmic drops, ophthalmic ointments, ophthalmic gels, powders, aerosols, transdermal modalities such as topical liquids, patches, creams and ointments, parenteral formulations and suppositories.

In embodiments, methods of treating an eye disorder are provided which include administering to a subject in need thereof a pharmaceutical composition including (1S,3S)-3-amino-4-difluoromethylenyl-1-cyclopentanoic acid or a pharmaceutically acceptable salt thereof wherein the composition provides improvement in one or more symptoms of the eye disorder for more than 1 hour after administration to the subject. In embodiments, methods of treating an eye disorder are provided which include administering to a subject in need thereof a pharmaceutical composition including (1S,3S)-3-amino-4-difluoromethylenyl-1-cyclopentanoic acid or a pharmaceutically acceptable salt thereof wherein the composition provides improvement in one or more symptoms of eye disorder for more than 2 hours after administration to the subject. In embodiments, methods of treating an eye disorder are provided which include administering to a subject in need thereof a pharmaceutical composition including (1S,3S)-3-amino-4-difluoromethylenyl-1-cyclopentanoic acid or a pharmaceutically acceptable salt thereof wherein the composition provides improvement in one or more symptoms of the eye disorder for more than 3 hours after administration to the subject. In embodiments, methods of treating an eye disorder are provided which include administering to a subject in need thereof a pharmaceutical composition including (1S,3S)-3-amino-4-difluoromethylenyl-1-cyclopentanoic acid or a pharmaceutically acceptable salt thereof wherein the composition provides improvement in one or more symptoms of the eye disorder for more than 4 hours after administration to the subject. In embodiments, methods of treating an eye disorder are provided which include administering to a subject in need thereof a pharmaceutical composition including (1S,3S)-3-amino-4-difluoromethylenyl-1-cyclopentanoic acid or a pharmaceutically acceptable salt thereof wherein the composition provides improvement in one or more symptoms of the eye disorder for more than 6 hours after administration to the subject. In embodiments, methods of treating an eye disorder are provided which include administering to a subject in need thereof a pharmaceutical composition including (1S,3S)-3-amino-4-difluoromethylenyl-1-cyclopentanoic acid or a pharmaceutically acceptable salt thereof wherein the composition provides improvement in one or more symptoms of the eye disorder for more than 8, 10, 12, 14, 16, 18, 20, 22 or 24 hours after administration to the subject. In embodiments, the pharmaceutical compositions provide improvement in one or more symptoms of the eye disorder the next day after administration to the subject. For example, the pharmaceutical compositions may provide improvement in one or more symptoms of the eye disorder for more than about, e.g., 2 hours, 4 hours, 6 hours, 8 hours, 10 hours, 12 hours, 14 hours, 16 hours, 18 hours, 20 hours, 22 hours or 24 hours after administration at bedtime or earlier, and waking from a night of sleep.

In embodiments, (1S,3S)-3-amino-4-difluoromethylenyl-1-cyclopentanoic acid or a pharmaceutically acceptable salt thereof is administered to a subject having an eye disorder in combination with (S)-3-amino-4-(difluoromethylenyl)cyclopent-1-ene-1-carboxylic acid or a pharmaceutically acceptable salt thereof. In embodiments, (1S,3S)-3-amino-4-difluoromethylenyl-1-cyclopentanoic acid or a pharmaceutically acceptable salt thereof, or (S)-3-amino-4-(difluoromethylenyl)cyclopent-1-ene-1-carboxylic acid or a pharmaceutically acceptable salt thereof, may be administered to a subject having an eye disorder in separate dosage forms or combined in one dosage form. In embodiments, (1S,3S)-3-amino-4-difluoromethylenyl-1-cyclopentanoic acid or a pharmaceutically acceptable salt thereof, or (S)-3-amino-4-(difluoromethylenyl)cyclopent-1-ene-1-carboxylic acid or a pharmaceutically acceptable salt thereof, may be administered to a subject having an eye disorder simultaneously or at spaced apart intervals.

In embodiments, methods include treating an eye disorder by administering to a subject in need thereof about 0.005 mg to about 750 mg of (S)-3-amino-4-(difluoromethylenyl)cyclopent-1-ene-1-carboxylic acid or a pharmaceutically acceptable salt thereof, e.g., hydrochloride salt. In embodiments, the amount of (S)-3-amino-4-(difluoromethylenyl)cyclopent-1-ene-1-carboxylic acid or a pharmaceutically acceptable salt thereof, e.g., hydrochloride salt, can be between 0.005 and 1000 mg/day, or 0.005 mg/kg/day to 14 mg/kg/day, for treatment of an eye disorder. Such eye disorders are Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen (RPD), eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy (APMPPE), acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome.

In embodiments, methods include treating Stargardt disease by administering to a subject in need thereof about 0.005 mg to about 750 mg of (S)-3-amino-4-(difluoromethylenyl)cyclopent-1-ene-1-carboxylic acid or a pharmaceutically acceptable salt thereof, e.g., hydrochloride salt. In embodiments, the amount of (S)-3-amino-4-(difluoromethylenyl)cyclopent-1-ene-1-carboxylic acid or a pharmaceutically acceptable salt thereof, e.g., hydrochloride salt, can be between 0.005 and 1000 mg/day, or 0.005 mg/kg/day to 14 mg/kg/day, for treatment of Stargardt disease. In embodiments, the amount of (S)-3-amino-4-(difluoromethylenyl)cyclopent-1-ene-1-carboxylic acid or a pharmaceutically acceptable salt thereof, e.g., hydrochloride salt, can be between 0.02 and 0.2 mg/kg/day for treatment of Stargardt disease. In embodiments, the amount of (S)-3-amino-4-(difluoromethylenyl)cyclopent-1-ene-1-carboxylic acid or a pharmaceutically acceptable salt thereof, e.g., hydrochloride salt, can be between 0.5 and 50 mg/day for treatment of Stargardt disease. In embodiments, the maximum amount of (S)-3-amino-4-(difluoromethylenyl)cyclopent-1-ene-1-carboxylic acid or a pharmaceutically acceptable salt thereof, e.g., hydrochloride salt, can be 75 mg/day for treatment of Stargardt disease.

For example, in embodiments, the daily dosage of (S)-3-amino-4-(difluoromethylenyl)cyclopent-1-ene-1-carboxylic acid or a pharmaceutically acceptable salt thereof can be, e.g., in the range of about 0.01 to 750 mg, 0.01 to 700 mg, 0.01 to 500 mg, 0.01 to 250 mg, 0.01 to 200 mg, 0.01 to 175 mg, 0.01 to 150 mg, 0.01 to 125 mg, 0.01 to 100 mg, 0.01 to 75 mg, 0.01 to 50 mg, 0.01 to 30 mg, 0.01 to 25 mg, 0.01 to 20 mg, 0.01 to 15 mg, 0.01 to 10 mg, 0.01 to 5 mg, 0.01 to 4 mg, 0.01 to 3 mg, 0.01 to 2 mg, 0.01 to 1 mg, 0.01 to 0.75 mg, 0.01 to 0.5 mg, 0.01 to 0.25 mg, 0.01 to 0.1 mg, 0.1 to 750 mg, 0.1 to 700 mg, 0.1 to 500 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 4 mg, 0.1 to 3 mg, 0.1 to 2 mg, 0.1 to 1 mg, 0.1 to 0.75 mg, 0.1 to 0.5 mg, 0.1 to 0.25 mg, 0.25 to 750 mg, 0.25 to 700 mg, 0.25 to 500 mg, 0.25 to 250 mg, 0.25 to 200 mg, 0.25 to 175 mg, 0.25 to 150 mg, 0.25 to 125 mg, 0.25 to 100 mg, 0.25 to 75 mg, 0.25 to 50 mg, 0.25 to 30 mg, 0.25 to 25 mg, 0.25 to 20 mg, 0.25 to 15 mg, 0.25 to 10 mg, 0.25 to 5 mg, 0.25 to 4 mg, 0.25 to 3 mg, 0.25 to 2 mg, 0.25 to 1 mg, 0.25 to 0.75 mg, 0.25 to 0.5 mg, 0.3 to 750 mg, 0.5 to 700 mg, 0.3 to 500 mg, 0.3 to 250 mg, 0.3 to 200 mg, 0.3 to 175 mg, 0.3 to 150 mg, 0.3 to 125 mg, 0.3 to 100 mg, 0.3 to 75 mg, 0.3 to 50 mg, 0.3 to 30 mg, 0.3 to 25 mg, 0.3 to 20 mg, 0.3 to 15 mg, 0.3 to 10 mg, 0.3 to 5 mg, 0.3 to 4 mg, 0.3 to 3 mg, 0.3 to 2 mg, 0.3 to 1 mg, 0.3 to 0.75 mg, 0.3 to 0.5 mg, 0.4 to 750 mg, 0.4 to 700 mg, 0.4 to 500 mg, 0.4 to 250 mg, 0.4 to 200 mg, 0.4 to 175 mg, 0.4 to 150 mg, 0.4 to 125 mg, 0.4 to 100 mg, 0.4 to 75 mg, 0.4 to 50 mg, 0.4 to 30 mg, 0.4 to 25 mg, 0.4 to 20 mg, 0.4 to 15 mg, 0.4 to 10 mg, 0.4 to 5 mg, 0.4 to 4 mg, 0.4 to 3 mg, 0.4 to 2 mg, 0.4 to 1 mg, 0.4 to 0.75 mg, 0.4 to 0.5 mg, 0.5 to 750 mg, 0.5 to 700 mg, 0.5 to 500 mg, 0.5 to 250 mg, 0.5 to 200 mg, 0.5 to 175 mg, 0.5 to 150 mg, 0.5 to 125 mg, 0.5 to 100 mg, 0.5 to 75 mg, 0.5 to 50 mg, 0.5 to 30 mg, 0.5 to 25 mg, 0.5 to 20 mg, 0.5 to 15 mg, 0.5 to 10 mg, 0.5 to 5 mg, 0.5 to 4 mg, 0.5 to 3 mg, 0.5 to 2 mg, 0.5 to 1 mg, 0.5 to 0.75 mg, 0.75 to 750 mg, 0.75 to 700 mg, 0.75 to 500 mg, 0.75 to 250 mg, 0.75 to 200 mg, 0.75 to 175 mg, 0.75 to 150 mg, 0.75 to 125 mg, 0.75 to 100 mg, 0.75 to 75 mg, 0.75 to 50 mg, 0.75 to 30 mg, 0.75 to 25 mg, 0.75 to 20 mg, 0.75 to 15 mg, 0.75 to 10 mg, 0.75 to 5 mg, 0.75 to 4 mg, 0.75 to 3 mg, 0.75 to 2 mg, 0.75 to 1 mg, 1 to 750 mg, 1 to 700 mg, 1 to 500 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 1 to 4 mg, 1 to 3 mg, 1 to 2 mg, 2 to 750 mg, 2 to 700 mg, 2 to 500 mg, 2 to 250 mg, 2 to 200 mg, 2 to 175 mg, 2 to 150 mg, 2 to 125 mg, 2 to 100 mg, 2 to 75 mg, 2 to 50 mg, 2 to 30 mg, 2 to 25 mg, 2 to 20 mg, 2 to 15 mg, 2 to 10 mg, 2 to 5 mg, 2 to 4 mg, 2 to 3 mg, 3 to 750 mg, 3 to 700 mg, 3 to 500 mg, 3 to 250 mg, 3 to 200 mg, 3 to 175 mg, 3 to 150 mg, 3 to 125 mg, 3 to 100 mg, 3 to 75 mg, 3 to 50 mg, 3 to 30 mg, 3 to 25 mg, 3 to 20 mg, 3 to 15 mg, 3 to 10 mg, 3 to 5 mg, 3 to 4 mg, 4 to 750 mg, 4 to 700 mg, 4 to 500 mg, 4 to 250 mg, 4 to 200 mg, 4 to 175 mg, 4 to 150 mg, 4 to 125 mg, 4 to 100 mg, 4 to 75 mg, 4 to 50 mg, 4 to 30 mg, 4 to 25 mg, 4 to 20 mg, 4 to 15 mg, 4 to 10 mg, 4 to 5 mg, 5 to 750 mg, 5 to 700 mg, 5 to 500 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 7.5 to 15 mg, 2.5 to 5 mg, with doses of, e.g., about 0.01 mg, 0.025 mg, 0.05 mg, 0.075 mg, 0.1 mg, 0.2 mg, 0.25 mg, 0.3 mg, 0.4 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 6 mg, 7 mg, 7.5 mg, 10 mg, 12.5 mg, 15 mg, 17.5 mg, 20 mg, 22.5 mg, 25 mg, 27.5 mg, 30 mg, 35 mg, 40 mg, 45 mg, 50 mg, 75 mg, 100 mg, 125 mg, 150 mg, 175 mg, 200 mg, 225 mg, 250 mg, 275 mg, 300 mg, 400 mg and 500 mg being examples.

In embodiments, pharmaceutical compositions may include (S)-3-amino-4-(difluoromethylenyl)cyclopent-1-ene-1-carboxylic acid or a pharmaceutically acceptable salt thereof in an amount of, e.g., about 0.001 to 500 mg, 0.01 to 500 mg, 0.01 to 450 mg, 0.01 to 300 mg, 0.01 to 250 mg, 0.01 to 200 mg, 0.01 to 175 mg, 0.01 to 150 mg, 0.01 to 125 mg, 0.01 to 100 mg, 0.01 to 75 mg, 0.01 to 50 mg, 0.01 to 30 mg, 0.01 to 25 mg, 0.01 to 20 mg, 0.01 to 15 mg, 0.01 to 10 mg, 0.01 to 5 mg, 0.01 to 1 mg, 0.025 to 500 mg, 0.025 to 450 mg, 0.025 to 300 mg, 0.025 to 250 mg, 0.025 to 200 mg, 0.025 to 175 mg, 0.025 to 150 mg, 0.025 to 125 mg, 0.025 to 100 mg, 0.025 to 75 mg, 0.025 to 50 mg, 0.025 to 30 mg, 0.025 to 25 mg, 0.025 to 20 mg, 0.025 to 15 mg, 0.025 to 10 mg, 0.025 to 5 mg, 0.025 to 1 mg, 0.05 to 500 mg, 0.05 to 450 mg, 0.05 to 300 mg, 0.05 to 250 mg, 0.05 to 200 mg, 0.05 to 175 mg, 0.05 to 150 mg, 0.05 to 125 mg, 0.05 to 100 mg, 0.05 to 75 mg, 0.05 to 50 mg, 0.05 to 30 mg, 0.05 to 25 mg, 0.05 to 20 mg, 0.05 to 15 mg, 0.05 to 10 mg, 0.05 to 5 mg, 0.05 to 1 mg, 0.075 to 500 mg, 0.075 to 450 mg, 0.075 to 300 mg, 0.075 to 250 mg, 0.075 to 200 mg, 0.075 to 175 mg, 0.075 to 150 mg, 0.075 to 125 mg, 0.075 to 100 mg, 0.075 to 75 mg, 0.075 to 50 mg, 0.075 to 30 mg, 0.075 to 25 mg, 0.075 to 20 mg, 0.075 to 15 mg, 0.075 to 10 mg, 0.075 to 5 mg, 0.075 to 1 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 0.25 to 500 mg, 0.25 to 450 mg, 0.25 to 300 mg, 0.25 to 250 mg, 0.25 to 200 mg, 0.25 to 175 mg, 0.25 to 150 mg, 0.25 to 125 mg, 0.25 to 100 mg, 0.25 to 75 mg, 0.25 to 50 mg, 0.25 to 30 mg, 0.25 to 25 mg, 0.25 to 20 mg, 0.25 to 15 mg, 0.25 to 10 mg, 0.25 to 5 mg, 0.25 to 1 mg, 0.05 to 500 mg, 0.5 to 450 mg, 0.5 to 300 mg, 0.5 to 250 mg, 0.5 to 200 mg, 0.5 to 175 mg, 0.5 to 150 mg, 0.5 to 125 mg, 0.5 to 100 mg, 0.5 to 75 mg, 0.5 to 50 mg, 0.5 to 30 mg, 0.5 to 25 mg, 0.5 to 20 mg, 0.5 to 15 mg, 0.5 to 10 mg, 0.5 to 5 mg, 0.5 to 1 mg, 1 to 500 mg, 1 to 450 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 1 to 4 mg, 1 to 3 mg, 1 to 2 mg, 2 to 500 mg, 2 to 450 mg, 2 to 300 mg, 2 to 250 mg, 2 to 200 mg, 2 to 175 mg, 2 to 150 mg, 2 to 125 mg, 2 to 100 mg, 2 to 75 mg, 2 to 50 mg, 2 to 30 mg, 2 to 25 mg, 2 to 20 mg, 2 to 15 mg, 2 to 10 mg, 2 to 5 mg, 3 to 500 mg, 3 to 450 mg, 3 to 300 mg, 3 to 250 mg, 3 to 200 mg, 3 to 175 mg, 3 to 150 mg, 3 to 125 mg, 3 to 100 mg, 3 to 75 mg, 3 to 50 mg, 3 to 30 mg, 3 to 25 mg, 3 to 20 mg, 3 to 15 mg, 3 to 10 mg, 3 to 5 mg, 4 to 500 mg, 4 to 450 mg, 4 to 300 mg, 4 to 250 mg, 4 to 200 mg, 4 to 175 mg, 4 to 150 mg, 4 to 125 mg, 4 to 100 mg, 4 to 75 mg, 4 to 50 mg, 4 to 30 mg, 4 to 25 mg, 4 to 20 mg, 4 to 15 mg, 4 to 10 mg, 4 to 5 mg, 5 to 500 mg, 5 to 450 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 500 mg, 10 to 450 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 500 mg, 15 to 450 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 500 mg, 20 to 450 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 500 mg, 25 to 450 mg, 25 to 300 mg, 25 to 250 mg, 25 to 200 mg, 25 to 175 mg, 25 to 150 mg, 25 to 125 mg, 25 to 100 mg, 25 to 80 mg, 25 to 75 mg, 25 to 50 mg, 25 to 30 mg, 30 to 500 mg, 30 to 450 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 40 to 500 mg, 40 to 450 mg, 40 to 400 mg, 40 to 250 mg, 40 to 200 mg, 40 to 175 mg, 40 to 150 mg, 40 to 125 mg, 40 to 100 mg, 40 to 75 mg, 40 to 50 mg, 50 to 500 mg, 50 to 450 mg, 50 to 300 mg, 50 to 250 mg, 50 to 200 mg, 50 to 175 mg, 50 to 150 mg, 50 to 125 mg, 50 to 100 mg, 50 to 75 mg, 75 to 500 mg, 75 to 450 mg, 75 to 300 mg, 75 to 250 mg, 75 to 200 mg, 75 to 175 mg, 75 to 150 mg, 75 to 125 mg, 75 to 100 mg, 100 to 500 mg, 100 to 450 mg, 100 to 300 mg, 100 to 250 mg, 100 to 200 mg, 100 to 175 mg, 100 to 150 mg, 100 to 125 mg, 125 to 500 mg, 125 to 450 mg, 125 to 300 mg, 125 to 250 mg, 125 to 200 mg, 125 to 175 mg, 125 to 150 mg, 150 to 500 mg, 150 to 450 mg, 150 to 300 mg, 150 to 250 mg, 150 to 200 mg, 200 to 500 mg, 200 to 450 mg, 200 to 300 mg, 200 to 250 mg, 250 to 500 mg, 250 to 450 mg, 250 to 300 mg, 300 to 500 mg, 300 to 450 mg, 300 to 400 mg, 300 to 350 mg, 350 to 500 mg, 350 to 450 mg, 350 to 400 mg, 400 to 500 mg, 400 to 450 mg, with 0.01 mg, 0.025 mg, 0.05 mg, 0.075 mg, 0.1 mg, 0.25 mg, 0.5 mg, 0.75 mg, 1 mg, 2 mg, 2.5 mg, 3 mg, 4 mg, 5 mg, 7.5 mg, 10 mg, 12.5 mg, 15 mg, 17.5 mg, 20 mg, 22.5 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg 175 mg, 200 mg, 225 mg, 250 mg, 275 mg, 300 mg, 325 mg, 350 mg, 375 mg, 400 mg, 425 mg, 450 mg, 475 mg, and 500 mg being examples.

Typically, dosages for treating Stargardt disease may be administered to a subject once, twice, three or four times daily, every other day, once weekly, or once a month. In embodiments, (S)-3-amino-4-(difluoromethylenyl) cyclopent-1-ene-1-carboxylic acid or a pharmaceutically acceptable salt thereof is administered to a subject twice a day, (e.g., morning and evening), or three times a day (e.g., at breakfast, lunch, and dinner), at a dose of 0.01-50 mg/administration. In embodiments, (S)-3-amino-4-(difluoromethylenyl)cyclopent-1-ene-1-carboxylic acid or a pharmaceutically acceptable salt thereof is administered to a subject 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 7.5 mg/per day, 5.5 mg/per day, 5 mg/per day, 4.5 mg/per day, 4 mg/per day, 3.5 mg/per day, 3 mg/per day, 2.5 mg/per day, 2 mg/per day, 1.5 mg/per day, 1 mg/per day, 0.5 mg/per day, 0.25 mg/per day, in one or more doses. In embodiments, an adult dose for treating Stargardt disease can be about 0.5 to 50 mg per day and can be increased to 75 mg per day. Dosages can be lower for children than for adults. In embodiments, a pediatric dose for treating Stargardt disease can be from about 0.01 to 10 mg per day once or in 2, 3 or 4 divided doses. In embodiments, a pediatric dose for treating Stargardt disease can be 0.075 mg/kg/day to 1.0 mg/kg/day. In embodiments, the subject may be started at a low dose and the dosage is escalated over time.

Typically, dosages for treating one or more of the following eye disorders: macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome, may be administered to a subject once, twice, three or four times daily, every other day, once weekly, or once a month. In embodiments, (S)-3-amino-4-(difluoromethylenyl) cyclopent-1-ene-1-carboxylic acid or a pharmaceutically acceptable salt thereof is administered to a subject twice a day, (e.g., morning and evening), or three times a day (e.g., at breakfast, lunch, and dinner), at a dose of 0.01-50 mg/administration. In embodiments, (S)-3-amino-4-(difluoromethylenyl)cyclopent-1-ene-1-carboxylic acid or a pharmaceutically acceptable salt thereof is administered to a subject 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 7.5 mg/per day, 5.5 mg/per day, 5 mg/per day, 4.5 mg/per day, 4 mg/per day, 3.5 mg/per day, 3 mg/per day, 2.5 mg/per day, 2 mg/per day, 1.5 mg/per day, 1 mg/per day, 0.5 mg/per day, 0.25 mg/per day, in one or more doses. In embodiments, an adult dose for treating Stargardt disease can be about 0.5 to 50 mg per day and can be increased to 75 mg per day. Dosages can be lower for children than for adults. In embodiments, a pediatric dose for treating an eye disease can be from about 0.01 to 10 mg per day once or in 2, 3 or 4 divided doses. In embodiments, a pediatric dose for treating an eye disease can be from about 0.01 to 2 mg per day once or in 2, 3 or 4 divided doses. In embodiments, a pediatric dose for treating an eye disease can be 0.01 mg/kg/day to 1.0 mg/kg/day. In embodiments, a pediatric dose for treating an eye disease can be 0.02 mg/kg/day to 0.2 mg/kg/day. In embodiments, the subject may be started at a low dose and the dosage is escalated over time.

In embodiments, (S)-3-amino-4-(difluoromethylenyl)cyclopent-1-ene-1-carboxylic acid or a pharmaceutically acceptable salt thereof is administered via a pharmaceutical composition. In embodiments, as discussed below, various dosage forms including conventional formulations and modified release formulations containing (S)-3-amino-4-(difluoromethylenyl)cyclopent-1-ene-1-carboxylic acid or a pharmaceutically acceptable salt thereof can be administered one or more times daily. Any suitable route of administration may be utilized, e.g., oral, rectal, nasal, ophthalmic, pulmonary, vaginal, sublingual, transdermal, intravenous, intraarterial, intramuscular, intraperitoneal and subcutaneous routes. Suitable dosage forms include tablets, capsules, oral liquids, ophthalmic drops, ophthalmic ointments, ophthalmic gels, powders, aerosols, transdermal modalities such as topical liquids, patches, creams and ointments, parenteral formulations and suppositories.

In embodiments, methods of treating an eye disorder are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more symptoms of the eye disorder for more than 1 hour after administration to the subject. In embodiments, methods of treating an eye disorder are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more symptoms of the eye disorder for more than 2 hours after administration to the subject. In embodiments, methods of treating an eye disorder are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more symptoms of the eye disorder for more than 3 hours after administration to the subject. In embodiments, methods of treating an eye disorder are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more symptoms of the eye disorder for more than 4 hours after administration to the subject. In embodiments, methods of treating an eye disorder are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more symptoms of the eye disorder for more than 6 hours after administration to the subject. In embodiments, methods of treating an eye disorder are provided which include administering to a subject in need thereof a pharmaceutical composition including (S)-3-amino-4-(difluoromethylenyl)cyclopent-1-ene-1-carboxylic acid or a pharmaceutically acceptable salt thereof wherein the composition provides improvement in one or more symptoms of the eye disorder for more than 8, 10, 12, 14, 16, 18, 20, 22 or 24 hours after administration to the subject. In embodiments, the pharmaceutical compositions provide improvement of next day symptoms of the subject. For example, the pharmaceutical compositions may provide improvement in one or more symptoms of the eye disorder for more than about, e.g., 2 hours, 4 hours, 6 hours, 8 hours, 10 hours, 12 hours, 14 hours, 16 hours, 18 hours, 20 hours, 22 hours or 24 hours after administration at bedtime or earlier, and waking from a night of sleep.

In embodiments, (S)-3-amino-4-(difluoromethylenyl)cyclopent-1-ene-1-carboxylic acid or a pharmaceutically acceptable salt thereof or a pharmaceutically acceptable salt thereof is administered to a subject having an eye disorder in combination with (1S,3S)-3-amino-4-difluoromethylenyl-1-cyclopentanoic acid or a pharmaceutically acceptable salt thereof. In embodiments, (S)-3-amino-4-(difluoromethylenyl)cyclopent-1-ene-1-carboxylic acid or a pharmaceutically acceptable salt thereof, or (1S,3S)-3-amino-4-difluoromethylenyl-1-cyclopentanoic acid or a pharmaceutically acceptable salt thereof, may be administered to a subject having an eye disorder in separate dosage forms or combined in one dosage form. In embodiments, (S)-3-amino-4-(difluoromethylenyl)cyclopent-1-ene-1-carboxylic acid or a pharmaceutically acceptable salt thereof, or (1S,3S)-3-amino-4-difluoromethylenyl-1-cyclopentanoic acid or a pharmaceutically acceptable salt thereof, may be administered to a subject having an eye disorder simultaneously or at spaced apart intervals.

In embodiments, provided herein are methods of treating an eye disorder including administering to a subject in need thereof (1 S,3S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid or a pharmaceutically acceptable salt of any of the preceding, which provides an in vivo plasma profile, wherein the in vivo plasma profile of the subject 10 hours after administration of (1 S,3 S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid or a pharmaceutically acceptable salt of any of the preceding, is reduced by more than 50% and the method provides improvement in the subject for more than 10, 12, 14, 16, 18, 20, 22 or 24 hours after administration. In embodiments, provided herein are methods of treating an eye disorder including administering to a subject in need thereof (S)-3-amino-4-(difluoromethylenyl)cyclopent-1-ene-1-carboxylic acid or a pharmaceutically acceptable salt thereof, either alone or in combination with (1 S,3 S)-3-amino-4-(difluoromethylidene)cyclopentane-1-carboxylic or a pharmaceutically acceptable salt of any of the preceding, which provides an in vivo plasma profile, wherein the in vivo plasma profile of the subject 10 hours after administration of (S)-3-amino-4-(difluoromethylenyl)cyclopent-1-ene-1-carboxylic acid or a pharmaceutically acceptable salt thereof, either alone or in combination with (1S,3S)-3-amino-4-(difluoromethylidene)cyclopentane-1-carboxylic acid or a pharmaceutically acceptable salt of any of the preceding, is reduced by more than 50% and the method provides improvement in the subject for more than 10, 12, 14, 16, 18, 20, 22 or 24 hours after administration.

In embodiments, provided herein are methods of treating an eye disorder including administering to a subject in need thereof (1 S,3S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid, or a pharmaceutically acceptable salt of any of the preceding, which provides an in vivo plasma profile, wherein the in vivo plasma profile of the subject 10 hours after administration of (1 S,3 S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid or a pharmaceutically acceptable salt of any of the preceding, is reduced by more than 55% and the method provides improvement in the subject for more than 10, 12, 14, 16, 18, 20, 22 or 24 hours after administration. In embodiments, provided herein are methods of treating an eye disorder including administering to a subject in need thereof (S)-3-amino-4-(difluoromethylenyl)cyclopent-1-ene-1-carboxylic acid or a pharmaceutically acceptable salt thereof, either alone or in combination with (1 S,3 S)-3-amino-4-(difluoromethylidene)cyclopentane-1-carboxylic acid or a pharmaceutically acceptable salt of any of the preceding, which provides an in vivo plasma profile, wherein the in vivo plasma profile of the subject 10 hours after administration of (S)-3-amino-4-(difluoromethylenyl)cyclopent-1-ene-1-carboxylic acid or a pharmaceutically acceptable salt thereof, either alone or in combination with (1S,3S)-3-amino-4-(difluoromethylidene)cyclopentane-1-carboxylic acid or a pharmaceutically acceptable salt of any of the preceding, is reduced by more than 55% and the method provides improvement in the subject for more than 10, 12, 14, 16, 18, 20, 22 or 24 hours after administration.

In embodiments, provided herein are methods of treating an eye disorder including administering to a subject in need thereof (1 S,3S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid or a pharmaceutically acceptable salt of any of the preceding, which provides an in vivo plasma profile, wherein the in vivo plasma profile of the subject 10 hours after administration of (1 S,3 S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid or a pharmaceutically acceptable salt of any of the preceding, is reduced by more than 60% and the method provides improvement in the subject for more than 10, 12, 14, 16, 18, 20, 22 or 24 hours after administration. In embodiments, provided herein are methods of treating an eye disorder including administering to a subject in need thereof (S)-3-amino-4-(difluoromethylenyl)cyclopent-1-ene-1-carboxylic acid or a pharmaceutically acceptable salt thereof, either alone or in combination with (1 S,3 S)-3-amino-4-(difluoromethylidene)cyclopentane-1-carboxylic acid or a pharmaceutically acceptable salt of any of the preceding, which provides an in vivo plasma profile, wherein the in vivo plasma profile of the subject 10 hours after administration of (S)-3-amino-4-(difluoromethylenyl)cyclopent-1-ene-1-carboxylic acid or a pharmaceutically acceptable salt thereof, either alone or in combination with (1 S,3 S)-3-amino-4-(difluoromethylidene)cyclopentane-1-carboxylic acid or a pharmaceutically acceptable salt of any of the preceding, is reduced by more than 60% and the method provides improvement in the subject for more than 10, 12, 14, 16, 18, 20, 22 or 24 hours after administration.

In embodiments, provided herein are methods of treating an eye disorder including administering to a subject in need thereof (1 S,3S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid or a pharmaceutically acceptable salt of any of the preceding, which provides an in vivo plasma profile, wherein the in vivo plasma profile of the subject 10 hours after administration of (1 S,3 S)-3-amino-4-(difluoromethylidene)cyclopentane-1-carboxylic acid or a pharmaceutically acceptable salt thereof, either alone or in combination with (S)-3-amino-4-(difluoromethylenyl)cyclopent-1-ene-1-carboxylic acid or a pharmaceutically acceptable salt of any of the preceding, is reduced by more than 65% and the method provides improvement in the subject for more than 10, 12, 14, 16, 18, 20, 22 or 24 hours after administration. In embodiments, provided herein are methods of treating an eye disorder including administering to a subject in need thereof (S)-3-amino-4-(difluoromethylenyl)cyclopent-1-ene-1-carboxylic acid or a pharmaceutically acceptable salt thereof, either alone or in combination with (1 S,3 S)-3-amino-4-(difluoromethylidene)cyclopentane-1-carboxylic acid or a pharmaceutically acceptable salt of any of the preceding, which provides an in vivo plasma profile, wherein the in vivo plasma profile of the subject 10 hours after administration of (S)-3-amino-4-(difluoromethylenyl)cyclopent-1-ene-1-carboxylic acid or a pharmaceutically acceptable salt thereof, either alone or in combination with (1S,3S)-3-amino-4-(difluoromethylidene)cyclopentane-1-carboxylic acid or a pharmaceutically acceptable salt of any of the preceding, is reduced by more than 65% and the method provides improvement in the subject for more than 10, 12, 14, 16, 18, 20, 22 or 24 hours after administration.

In embodiments, provided herein are methods of an eye disorder wherein the amount of active substance, e.g., (1 S,3 S)-3-amino-4-(difluoromethylidene)cyclopentane-1-carboxylic acid, or (S)-3-amino-4-(difluoromethylenyl)cyclopent-1-ene-1-carboxylic acid (KT-II-115), or a pharmaceutically acceptable salt of any of the preceding, individually or in any combination, within the subject about 4 hours after administration of the pharmaceutical composition is less than about 75% of the administered dose. In embodiments, provided herein are methods wherein the amount of (1 S, 3 S)-3-amino-4-(difluoromethylidene)cyclopentane-1-carboxylic acid, or (S)-3-amino-4-(difluoromethylenyl)cyclopent-1-ene-1-carboxylic acid (KT-II-115), or a pharmaceutically acceptable salt of any of the preceding, individually or in any combination, within the subject about, e.g., 6 hours, 8 hours, 10 hours, 12 hours, 15 hours, or 20 hours after administration of the pharmaceutical composition is less than about 75%.

In embodiments, provided herein are methods of treating eye disorder wherein the amount of active substance, e.g., (1S,3S)-3-amino-4-(difluoromethylidene)cyclopentane-1-carboxylic acid, or (S)-3-amino-4-(difluoromethylenyl)cyclopent-1-ene-1-carboxylic acid (KT-II-115), or a pharmaceutically acceptable salt of any of the preceding, individually or in any combination, within the subject about 4 hours after administration of the pharmaceutical composition is less than about 80% of the administered dose. In embodiments, provided herein are methods wherein the amount of active substance, e.g., (1 S,3 S)-3-amino-4-(difluoromethylidene)cyclopentane-1-carboxylic acid, or (S)-3-amino-4-(difluoromethylenyl)cyclopent-1-ene-1-carboxylic acid (KT-II-115), or a pharmaceutically acceptable salt of any of the preceding, individually or in any combination, within the subject about, e.g., 6 hours, 8 hours, 10 hours, 12 hours, 15 hours, or 20 hours after administration of the pharmaceutical composition is less than about 80% of the administered dose.

In embodiments, provided herein are methods of treating an eye disorder wherein the amount of active substance, e.g., (1S,3S)-3-amino-4-(difluoromethylidene)cyclopentane-1-carboxylic acid, or (S)-3-amino-4-(difluoromethylenyl)cyclopent-1-ene-1-carboxylic acid (KT-II-115), or a pharmaceutically acceptable salt of any of the preceding, individually or in any combination, within the subject about 4 hours after administration of the pharmaceutical composition is between about 65% to about 85% of the administered dose. In embodiments, the amount of active substance, e.g., (1S,3S)-3-amino-4-(difluoromethylidene)cyclopentane-1-carboxylic acid, or (S)-3-amino-4-(difluoromethylenyl)cyclopent-1-ene-1-carboxylic acid (KT-II-115), or a pharmaceutically acceptable salt of any of the preceding, individually or in any combination, within the subject after about, e.g., 6 hours, 8 hours, 10 hours, 12 hours, 15 hours, or 20 hours after administration of the pharmaceutical composition is between about 65% to about 85% of the administered dose.

In embodiments, the pharmaceutical compositions described herein may be administered once daily, twice daily, three times daily, four times daily, or every other day. In embodiments, the pharmaceutical compositions described herein may be administered by continuous infusion. In embodiments, a pharmaceutical composition described herein is provided to the subject in the morning. In embodiments, a pharmaceutical composition described herein is provided to the subject in the evening. In embodiments, a pharmaceutical composition described herein is provided to the subject once in the evening and once in the morning. In embodiments, a pharmaceutical composition described herein is provided to the subject once in the morning, once in the afternoon and once in the evening.

In embodiments, as mentioned previously, pharmaceutical compositions herein may be provided with conventional release or modified release profiles. Pharmaceutical compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers.

In embodiments, pharmaceutical compositions herein are modified release dosage forms which provide modified release profiles. Modified release profiles may exhibit immediate release, delayed release, or extended release profiles. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Some subjects with an eye disorder may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which one or both of (1S,3S)-3-amino-4-(difluoromethylidene)cyclopentane-1-carboxylic acid, (S)-3-amino-4-(difluoromethylenyl)cyclopent-1-ene-1-carboxylic acid (KT-II-115), or a pharmaceutically acceptable salt of any of the preceding, is applied to beads, e.g., confectioners nonpareil beads, and then coated with conventional release retarding materials such as waxes, enteric coatings and the like. In embodiments, beads can be formed in which one or both of (1S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

Delayed release dosage forms are known to those skilled in the art. For example, coated delayed release beads or granules in which one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, is applied to beads, e.g., confectioners nonpareil beads, and then coated with conventional release delaying materials such as waxes, enteric coatings and the like. In embodiments, beads can be formed in which one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, one both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, is incorporated into porous inert carriers that provide delayed release profiles. In embodiments, the porous inert carriers incorporate channels or passages from which the drug diffuses into surrounding fluids. In embodiments, one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, pharmaceutical compositions described herein are suitable for ophthalmic or parenteral administration, including, e.g., intramuscular (i.m.), intravenous (i.v.), subcutaneous (s.c.), intraperitoneal (i.p.), or intrathecal (i.t.). Parenteral or ophthalmic compositions must be sterile for administration by injection, infusion, instillation or implantation into the body and may be packaged in either single-dose or multi-dose containers. In embodiments, liquid pharmaceutical compositions for ophthalmic or parenteral administration to a subject include an active substance, e.g., (1 S,3 S)-3-amino-4-(difluoromethylidene)cyclopentane-1-carboxylic acid, (S)-3-amino-4-(difluoromethylenyl)cyclopent-1-ene-1-carboxylic acid (KT-II-115), or a pharmaceutically acceptable salt of any of the preceding, in any of the respective amounts described above. In embodiments, the pharmaceutical compositions for ophthalmic or parenteral administration are formulated as a total volume of about, e.g., 1 ml, 2 ml, 3 ml, 4 ml, 5 ml, 7.5 ml, 10 ml, 20 ml, 25 ml, 50 ml, 100 ml, 200 ml, 250 ml, or 500 ml. In embodiments, the compositions are contained in a bag, a glass vial, a plastic vial, or a bottle.

In embodiments, pharmaceutical compositions for ophthalmic or parenteral administration include respective amounts described above for (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding. In embodiments, pharmaceutical compositions for ophthalmic or parenteral administration include about 0.05 mg to about 500 mg active substance, e.g., (1 S,3 S)-3-amino-4-(difluoromethylidene)cyclopentane-1-carboxylic acid or (S)-3-amino-4-(difluoromethylenyl)cyclopent-1-ene-1-carboxylic acid, or a pharmaceutically acceptable salt of any of the preceding. In embodiments, pharmaceutical compositions for ophthalmic or parenteral administration to a subject include an active substance, e.g., (1 S,3 S)-3-amino-4-(difluoromethylidene)cyclopentane-1-carboxylic acid or (S)-3-amino-4-(difluoromethylenyl)cyclopent-1-ene-1-carboxylic acid, or a pharmaceutically acceptable salt of any of the preceding, at a respective concentration of about 0.005 mg/ml to about 500 mg/ml. In embodiments, the pharmaceutical composition for ophthalmic or parenteral administration includes an active substance, e.g., (1S,3S)-3-amino-4-(difluoromethylidene)cyclopentane-1-carboxylic acid or (S)-3-amino-4-(difluoromethylenyl)cyclopent-1-ene-1-carboxylic acid, or a pharmaceutically acceptable salt of any of the preceding, at a respective concentration of, e.g., about 0.05 mg/ml to about 50 mg/ml, about 0.1 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the pharmaceutical composition for ophthalmic or parenteral administration includes an active substance, e.g., (1 S,3 S)-3-amino-4-(difluoromethylidene)cyclopentane-1-carboxylic acid or (5)-3-amino-4-(difluoromethylenyl)cyclopent-1-ene-1-carboxylic acid (KT-II-115), or a pharmaceutically acceptable salt of any of the preceding, at a respective concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml.

In embodiments, a pharmaceutical composition for ophthalmic or parenteral administration is provided wherein the pharmaceutical composition is stable for at least six months. In embodiments, the pharmaceutical compositions for ophthalmic or parenteral administration exhibit no more than about 5% decrease in active substance, e.g., (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, e.g., 3 months or 6 months. In embodiments, the amount of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for ophthalmic or parenteral administration are provided wherein the pharmaceutical composition remains soluble. In embodiments, pharmaceutical compositions for ophthalmic or parenteral administration are provided that are stable, soluble, local site compatible and/or ready-to-use. In embodiments, the pharmaceutical compositions herein are ready-to-use for direct administration to a subject in need thereof.

The pharmaceutical compositions for ophthalmic or parenteral administration provided herein may include one or more excipients, e.g., solvents, solubility enhancers, suspending agents, buffering agents, isotonicity agents, stabilizers or antimicrobial preservatives. When used, the excipients of the ophthalmic or parenteral compositions will not adversely affect the stability, bioavailability, safety, and/or efficacy of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, used in the composition. Thus, ophthalmic or parenteral compositions are provided wherein there is no incompatibility between any of the components of the dosage form.

In embodiments, ophthalmic or parenteral compositions including (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, include a stabilizing amount of at least one excipient. For example, excipients may be selected from the group consisting of buffering agents, solubilizing agents, tonicity agents, antioxidants, chelating agents, antimicrobial agents, and preservatives. One skilled in the art will appreciate that an excipient may have more than one function and be classified in one or more defined group.

In embodiments, ophthalmic or parenteral compositions including (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, ophthalmic or parenteral compositions may be administered as needed, e.g., once, twice, thrice or four or more times daily, or continuously depending on the subject's needs.

In embodiments, ophthalmic or parenteral compositions of an active substance, e.g., (1 S,3 S)-3-amino-4-(difluoromethylidene)cyclopentane-1-carboxylic acid or (S)-3-amino-4-(difluoromethylenyl)cyclopent-1-ene-1-carboxylic acid, or a pharmaceutically acceptable salt of any of the preceding, are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

In embodiments, provided herein are methods of treating an eye disorder such as Stargardt disease including administering to a subject in need thereof a pharmaceutical composition including an active substance, e.g., one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, in a respective amount described herein, wherein the composition provides an in vivo plasma profile having a Cₘₐₓ, individually or combined, less than about 800 ng/ml. In embodiments, the composition provides improvement for more than 6 hours after administration to the subject.

In embodiments, pharmaceutical compositions including one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, provide an in vivo plasma profile having a Cₘₐₓ, individually or combined, less than about, e.g., 2000 ng/ml, 1000 ng/ml, 850 ng/ml, 800 ng/ml, 750 ng/ml, 700 ng/ml, 650 ng/ml, 600 ng/ml, 550 ng/ml, 450 ng/ml, 400 ng/ml 350 ng/ml, or 300 ng/ml and wherein the composition provides improvement in symptoms of an eye disorder such as Stargardt disease the next day in the subject. In embodiments, the pharmaceutical composition provides an in vivo plasma profile having a Cₘₐₓ, individually or combined, less than about, e.g., 250 ng/ml, 200 ng/ml 150 ng/ml, or 100 ng/ml and wherein the composition provides improvement of symptoms of an eye disorder such as Stargardt disease in the subject. In embodiments, the pharmaceutical composition provides improvement in one or more symptoms of an eye disorder such as Stargardt disease for more than 6 hours after administration.

In embodiments, provided herein are methods of treating an eye disorder such as Stargardt disease including administering to a subject in need thereof a pharmaceutical composition containing one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides a consistent in vivo plasma profile having a AUC_{0-∞}, individually or combined, of less than about 900 ng·hr/ml. In embodiments, the pharmaceutical composition provides improvement in symptoms of an eye disorder such as Stargardt disease the next day. In embodiments, the compositions provide an in vivo plasma profile having a AUC_{0-∞}, individually or combined, of less than about, e.g., 850 ng·hr/ml, 800 ng·hr/ml, 750 ng·hr/ml, or 700 ng·hr/ml and wherein the pharmaceutical composition provides improvement in symptoms of an eye disorder such as Stargardt disease the next day in the subject. In embodiments, the composition provides improvement in one or more symptoms of an eye disorder such as Stargardt disease for more than 6 hours after administration.

In embodiments, provided herein are methods of treating an eye disorder such as Stargardt disease including administering to a subject in need thereof a pharmaceutical composition comprising an active substance, e.g., one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the pharmaceutical composition provides an in vivo plasma profile having a AUC_{0-∞}, individually or combined, of less than about, e.g., 650 ng·hr/ml, 600 ng·hr/ml, 550 ng·hr/ml, 500 ng·hr/ml, or 450 ng·hr/ml. In embodiments, the composition provides an in vivo plasma profile having a AUC_{0-∞}, individually or combined, of less than about, e.g., 400 ng·hr/ml, 350 ng·hr/ml, 300 ng·hr/ml, 250 ng·hr/ml, or 200 ng·hr/ml. In embodiments, the pharmaceutical composition provides an in vivo plasma profile having a AUC_{0-∞}, individually or combined, of less than about, e.g., 150 ng·hr/ml, 100 ng·hr/ml, 75 ng·hr/ml, or 50 ng·hr/ml. In embodiments, the pharmaceutical composition provides improvement in symptoms of an eye disorder such as Stargardt disease the next day in the subject, after administration for more than, e.g., 4 hours, 6 hours, 8 hours, 10 hours, or 12 hours, after administration of the composition to the subject.

In embodiments, provided herein are methods of treating an eye disorder such as Stargardt disease including administering to a subject in need thereof a first pharmaceutical composition including one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, and a second pharmaceutical composition including one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding.

In embodiments, the second pharmaceutical composition provides an in vivo plasma profile having a mean AUC_{0-∞} which is about the same as the mean AUC_{0-∞} of the first pharmaceutical composition. In embodiments, the second pharmaceutical composition provides an in vivo plasma profile having a mean AUC_{0-∞} of at least about 20% less than the first pharmaceutical composition. In embodiments, provided herein are methods of treating an eye disorder such as Stargardt disease including administering to a subject in need thereof a first pharmaceutical composition including one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid, or a pharmaceutically acceptable salt of any of the preceding, and a second pharmaceutical composition including one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the second pharmaceutical composition provides a stable in vivo plasma profile having a mean AUC_{0-∞} of at least about, e.g., 25%, 30%, 35%, 40%, 45% or 50% less than the first pharmaceutical composition. In embodiments, the compositions provide improvement in symptoms of an eye disorder such as Stargardt disease in the subject the next day after administration. In embodiments, the pharmaceutical compositions may provide improvement in one or more symptoms for more than about, e.g., 6 hours, 8 hours, 10 hours, 12 hours, 14 hours, 16 hours, 18 hours, 20 hours, 22 hours or 24 hours after administration of the first and/or second pharmaceutical composition.

In embodiments, provided herein are methods of treating an eye disorder such as Stargardt disease including administering to a subject in need thereof a first pharmaceutical composition including one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, and a second pharmaceutical composition including one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the second pharmaceutical composition provides an in vivo plasma profile having a mean AUC_{0-∞} of less than about 900 ng·hr/ml. In embodiments, the second pharmaceutical composition provides an in vivo plasma profile having a AUC_{0-∞} of less than about, e.g., 800 ng·hr/ml, 750 ng·hr/ml, 700 ng·hr/ml, 650 ng·hr/ml, or 600 ng·hr/ml. In embodiments, the second pharmaceutical composition provides an in vivo plasma profile having a AUC_{0-∞} of less than about, e.g., 550 ng·hr/ml, 500 ng·hr/ml, 450 ng·hr/ml, 400 ng·hr/ml, or 350 ng·hr/ml. In embodiments, the second pharmaceutical composition provides an in vivo plasma profile having a AUC_{0-∞} of less than about, e.g., 300 ng·hr/ml, 250 ng·hr/ml, 200 ng·hr/ml, 150 ng·hr/ml, or 100 ng·hr/ml. In embodiments, the first and second pharmaceutical composition are administered wherein the compositions provide improvement in symptoms of an eye disorder such as Stargardt disease the next day following administration in the subject. In embodiments, the first pharmaceutical composition provides improvement in one or more symptom for more than, e.g., 6 hours, 8 hours, 10 hours, 12 hours, 14 hours, 16 hours, 18 hours, 20 hours, 22 hours or 24 hours after administration of the first pharmaceutical composition.

In embodiments, provided herein are methods of treating an eye disorder such as Stargardt disease including administering to a subject in need thereof a first pharmaceutical composition including one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, and a second pharmaceutical composition including one or both of (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the first composition provides an in vivo plasma profile with a Cₘₐₓ that is more than about 50% greater than the Cₘₐₓ provided by the administration of the second pharmaceutical composition. As used herein the Cₘₐₓ provided by the administration of the second pharmaceutical composition may or may not include the plasma profile contribution of the first pharmaceutical composition. In embodiments, the administration of the second pharmaceutical composition does not include the plasma profile contribution of the first pharmaceutical composition. In embodiments, the first composition provides an in vivo plasma profile having a Cₘₐₓ that is more than about e.g., 60%, 70%, 80%, or 90% greater than the Cₘₐₓ provided by the administration of the second pharmaceutical composition.

In embodiments, the Tₘₐₓ of the first pharmaceutical composition is less than 3 hours. In embodiments, the Tₘₐₓ of the first pharmaceutical composition is less than 2.5 hours. In embodiments, the Tₘₐₓ of the first pharmaceutical composition is less than 2 hours. In embodiments, the Tₘₐₓ of the first pharmaceutical composition is less than 1.5 hours. In embodiments, the Tₘₐₓ of the first pharmaceutical composition is less than 1 hour. In embodiments, the Tₘₐₓ of the first pharmaceutical composition is less than 0.5 hour. In embodiments, the Tₘₐₓ of the first pharmaceutical composition is less than 0.25 hour. In embodiments, the Tₘₐₓ of the second pharmaceutical composition is less than 3 hours. In embodiments, the Tₘₐₓ of the second pharmaceutical composition is less than 2.5 hours. In embodiments, the Tₘₐₓ of the second pharmaceutical composition is less than 2 hours. In embodiments, the Tₘₐₓ of the second pharmaceutical composition is less than 1.5 hours. In embodiments, the Tₘₐₓ of the second pharmaceutical composition is less than 1 hour. In embodiments, the Tₘₐₓ of the second pharmaceutical composition is less than 0.5 hour. In embodiments, the Tₘₐₓ of the second pharmaceutical composition is less than 0.25 hour.

In embodiments, the first pharmaceutical composition provides a dissolution of at least about 80% within the first 20 minutes of administration to a subject in need thereof. In embodiments, the first pharmaceutical composition provides a dissolution of at least about, e.g., 85%, 90% or 95% within the first 20 minutes of administration to a subject in need thereof. In embodiments, the first pharmaceutical composition provides a dissolution of at least 80% within the first 10 minutes of administration to a subject in need thereof.

In embodiments, administration of the first and second pharmaceutical compositions may be simultaneous or separated by an interval of time to achieve long-term improvement in at least one symptom of an eye disorder such as Stargardt disease. In embodiments, the first and second pharmaceutical composition may be administered 1, 2, 3, 4, 5, 6, 7, 8, 9, 10, 11, or 12 hours apart. In embodiments the first and second pharmaceutical composition may be administered 12 hours apart. In embodiments, the first and second pharmaceutical compositions may administered within, e.g., 15 minutes, 30 minutes, 1 hour, 2 hours, 6 hours, 12 hours, 18 hours, 24 hours, etc. In embodiments, the first and second pharmaceutical compositions may administered separated by at least, e.g., 15 minutes, 30 minutes, 1 hour, 2 hours, 12 hours, 18 hours, 24 hours, etc. In embodiments, improvement in at least one symptom of an eye disorder such as Stargardt disease for more than 8 hours after administration to the subject is provided. In embodiments, improvement for more than about, e.g., 10 hours, 12 hours, 15 hours, 18 hours, 20 hours, 24 hours, 30 hours, 36 hours, 42 hours or 48 hours after administration to the subject is provided.

In embodiments, the administration of the first and second pharmaceutical composition may provide a synergistic effect to improve at least one symptom of an eye disorder such as Stargardt disease.

In embodiments, provided herein are methods of treating an eye disorder such as Stargardt disease including administering to a subject in need thereof a first pharmaceutical dosage including a sub-therapeutic amount of one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding. In embodiments, treating an eye disorder such as Stargardt disease includes administering to a subject in need thereof a first pharmaceutical dosage including a sub-therapeutic amount of one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides improvement in one or more symptoms of an eye disorder such as Stargardt disease for more than 6 hours after administration.

In embodiments, the first and/or the second pharmaceutical compositions contain sub-therapeutic dosages. A sub-therapeutic dosage is an amount of active substance, e.g., one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, that is less than the amount typically required for a therapeutic effect. In embodiments, a sub-therapeutic dosage is an amount of one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, that alone may not provide improvement in an eye disorder such as Stargardt disease but is sufficient to maintain an existing improvement from a previous administration of a therapeutic dose. In embodiments, the methods provide administering a first pharmaceutical composition that provides improvement in at least one symptom of an eye disorder such as Stargardt disease and a second composition that maintains the improvement. In embodiments, the second composition contains a sub-therapeutic dose of one or both of (1 S, 3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding. In embodiments, after administration of the first pharmaceutical composition, the second pharmaceutical composition may provide a synergistic effect to improve at least one symptom of an eye disorder such as Stargardt disease. In embodiments, the second pharmaceutical composition may provide a synergistic effect to improve at least one symptom of an eye disorder such as Stargardt disease.

In embodiments, provided herein are methods of treating an eye disorder such as Stargardt disease including administering to a subject in need thereof a first pharmaceutical composition including a first pharmaceutical dosage of, e.g., one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the first pharmaceutical dosage provides improvement for more than 6 hours after administration, and a second pharmaceutical composition including a sub-therapeutic dosage of one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding.

In embodiments, the first or the second pharmaceutical composition are provided to the subject once in the evening and once in the morning. In embodiments, the total amount of one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, administered to a subject in a 24-hour period is any of the respective amounts described herein.

In embodiments, the first and/or the second pharmaceutical compositions may be provided with conventional release or modified release profiles. The first and second pharmaceutical compositions may be provided at the same time or separated by an interval of time, e.g., 1 hour, 2 hours, 3 hours, 4 hours, 5 hours, 6 hours, 12 hours, etc. In embodiments, the first and the second pharmaceutical compositions may be provided with different drug release profiles to create a two-phase release profile. For example, the first pharmaceutical composition may be provided with an immediate release profile, e.g., ODDF, parenteral, etc., and the second pharmaceutical composition may provide an extended release profile. In embodiments, one or both of the first and second pharmaceutical compositions may be provided with an extended release or delayed release profile. Such compositions may be provided as pulsatile formulations, multilayer tablets or capsules containing tablets, beads, granules, etc. In embodiments, the first pharmaceutical composition is an immediate release composition. In embodiments, the second pharmaceutical composition is an immediate release composition. In embodiments, the first and second pharmaceutical compositions are provided as separate immediate release compositions, e.g., film, tablets or capsules. In embodiments the first and second pharmaceutical compositions are provided 12 hours apart.

It should be understood that respective dosage amounts of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, that are provided herein are applicable to all the dosage forms described herein including conventional dosage forms, modified dosage forms, the first and second pharmaceutical compositions, as well as the ophthalmic and parenteral formulations described herein. Those skilled in the art will determine appropriate amounts depending on criteria such as dosage form, route of administration, subject tolerance, efficacy, therapeutic goal and therapeutic benefit, among other pharmaceutically acceptable criteria.

Combination therapies utilizing one or both of (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, can include administration of the active agents together in the same admixture, or in separate admixtures. In embodiments, the pharmaceutical composition can includes two, three, or more active agents. In embodiments, the combinations result in a more than additive effect on the treatment of the disease or disorder. Thus, treatment is provided for an eye disorder such as Stargardt disease with a combination of agents that combined, may provide a synergistic effect that enhances efficacy.

In embodiments, a co-therapy of (1S,3S)-3-amino-4-(difluoromethylidene)cyclopentane-1-carboxylic acid and (S)-3-amino-4-(difluoromethylenyl)cyclopent-1-ene-1-carboxylic acid (KT-II-115), or a pharmaceutically acceptable salt of any of the preceding, is effective to reduce frequency or severity of symptoms in the subject greater than any of the compounds administered alone. In embodiments, the co-therapy produces a more than additive result compared to compounds administered individually.

In embodiments, the subject may be started at a low dose and the dosage is escalated. In this manner, it can be determined if the drug is well tolerated in the subject. Dosages can be lower for children than for adults.

In embodiments, provided herein are methods of treating an eye disorder such as Stargardt disease including administering to a patient in need thereof a pharmaceutical composition including (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, in combination with a second pharmaceutically active agent.

The second pharmaceutically active agent may include analgesics, local anesthetics, anti-inflammatory agents, anti-microbials, gabapentinoids and neuroprotectives. In embodiments analgesics may include opioids such as morphine, hydrocodone, oxycodone, codeine and fentanyl, non-steroidal analgesics such as acetaminophen. Gabapentinoids include gabapentin and pregabalin. Local anesthetics include benzocaine, prilocaine, lidocaine, cocaine and bupivacaine. Anti-inflammatory agents include corticosteroids and non-steroidal anti-inflammatory drugs such as ibuprofen, ketoprofen, sulindac, indomethacin, aspirin, and naproxen. Anti-inflammatory corticosteroids include prednisone, methylprednisolone, prednisolone, betamethasone, dexamethasone, fluorometholone, triamcinolone, fluticasone, fluocinolone and hydrocortisone. Antimicrobials include aminoglycosides such as gentamycin, neomycin and streptomycin, penicillins such as amoxicillin, ampicillin and penicillin, cephalosporins such as cephalexin, cefadroxyl, cefachlor, cefradine, cefepime, and cefpirome, glycopeptides such as vancomycin, macrolides such as erythromycin and azithromycin, tetracyclines such as tetracycline, minocycline and doxycycline, sulfonamides such as sulfsmethoxazole, fluoroquinones such as ciprofloxacin, antifungals such as clotrimazole, econazole, miconazole, terbinafine, fluconazole, ketoconazole, griseofulvin, nystatin and amphotericin, and antiseptics such as benzalkonium chloride.

The disclosed combinations may provide improved treatment compared to either active agent alone. For example, the combinations may provide synergy, e.g., low dose treatments may be particularly effective in reducing or eliminating symptoms of the eye disorders.

In embodiments, provided herein methods of treating an eye disorder such as Stargardt disease including administering to a patient in need thereof a pharmaceutical composition including (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides improvement in an eye disorder such as Stargardt disease the next day. In embodiments, provided herein methods of treating macular degeneration including administering to a patient in need thereof a pharmaceutical composition including (1 S,3 S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides improvement in macular degeneration the next day. In embodiments, provided herein methods of treating retinal dystrophy including administering to a patient in need thereof a pharmaceutical composition including (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides improvement in retinal dystrophy the next day. In embodiments, provided herein methods of treating juvenile retinoschisis including administering to a patient in need thereof a pharmaceutical composition including (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides improvement in juvenile retinoschisis the next day. In embodiments, provided herein methods of treating retinitis pigmentosa including administering to a patient in need thereof a pharmaceutical composition including (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides improvement in retinitis pigmentosa the next day. In embodiments, provided herein methods of treating Best disease including administering to a patient in need thereof a pharmaceutical composition including (1S,3S)-3-amino-4-(difluoromethylidene)cyclopentane-1-carboxylic acid or (S)-3-amino-4-(difluoromethylenyl)cyclopent-1-ene-1-carboxylic acid (KT-II-115), or a pharmaceutically acceptable salt of any of the preceding, wherein the composition provides improvement in Best disease the next day.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of an eye disorder such as Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome, relative to at least one symptom of the disorder.

“Improvement in one or more symptoms of an eye disorder a day after administration” refers to improvement wherein the beneficial effect of at least one symptom lasts over a period of time, e.g., 6 hours, 12 hours, 24 hours etc.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng·hr/ml). The area under the curve is governed by clearance. Clearance is defined as the volume of blood or plasma that is totally cleared of its content of drug per unit time (ml/min).

“Treating” or “treatment” refers to alleviating or delaying the appearance of clinical symptoms of a disease or condition in a subject that may be afflicted with or predisposed to the disease or condition, but does not yet experience or display clinical or subclinical symptoms of the disease or condition. In certain embodiments, “treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” also refers to inhibiting the disease or condition, e.g., arresting or reducing its development or at least one clinical or subclinical symptom thereof. “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate aspects of the disclosure herein.

“Pharmaceutically acceptable” refers to molecular entities and compositions that are “generally regarded as safe”—e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount” means a dosage sufficient to alleviate one or more symptoms of a disorder, disease, or condition being treated, e.g., Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome, or to otherwise provide a desired pharmacological and/or physiologic effect.

“Co-administered with”, “co-therapy”, “in combination with”, “a combination of”, “combined with” or “administered along with” may be used interchangeably and mean that two or more agents are administered in the course of therapy. The agents may be administered together at the same time or separately in spaced apart intervals. The agents may be administered in a single dosage form or in separate dosage forms.

“Patient in need thereof” includes individuals that have been diagnosed with an eye disorder such as Stargardt disease, macular degeneration, also known as age-related macular degeneration (AMD or ARMD), juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, and Wagner syndrome. The methods may be provided to any individual including, e.g., wherein the patient is a neonate, infant, a pediatric patient (6 months to 12 years), an adolescent patient (age 12-18 years) or an adult (over 18 years). “Patient” and “subject” are used interchangeably herein. It should be understood that infants can receive a pediatric dose.

### EXAMPLES

The Examples provided herein are included solely for augmenting the disclosure herein and should not be considered to be limiting in any respect.

### Example 1

Prospective Assessment of the Safety and Efficacy of (S)-3-amino-4-(difluoromethylenyl)cyclopent-1-ene-1-carboxylic Acid in Patients with Stargardt Disease

This study is designed to determine whether (S)-3-amino-4-(difluoromethylenyl)cyclopent-1-ene-1-carboxylic acid leads to an improvement in Stargardt Disease. The primary objective of this study may be to evaluate the safety and tolerability from Baseline to Week 6 and Week 12 of (S)-3-amino-4-(difluoromethylenyl)cyclopent-1-ene-1-carboxylic acid in subjects with Stargardt Disease across different dose levels and in two dosing schedules. The following dosing schedules may be tested against placebo: (1) Once daily (o.d.): An evening dose; and (2) Twice daily (b.i.d.): Evening and morning doses titrated to target doses.

The safety endpoints that relate to this study may include: (1) Frequency and severity of adverse events (AEs) and serious adverse events; (2) Vital signs (weight, blood pressure, temperature); (3) Laboratory parameters (electrolytes, lipids, glucose, liver and pancreas function tests, hematology, creatinine); (4) Suicidality assessed by ABC-Irritability Subscale; (5).

The secondary objective of this study may include the identification of a set of parameters that may best characterize the efficacy of (S)-3-amino-4-(difluoromethylenyl)cyclopent-1-ene-1-carboxylic acid in Stargardt Disease subjects for subsequent efficacy trials. These tests may be administered at four full day site visits (Screening, Baseline, Interim and End of Treatment) by an appropriately trained professional to provide the test to an adult Stargardt Disease patient. Assessments may be based in part on patient's perception of symptoms. Secondary outcome measures also involve 1. Spectral Domain-Optical Coherence Tomography (SD-OCT) [Time Frame: 6 months] Mean rate of change in the area of ellipsoid zone defect measured by en face SD-OCT and 2. Change in electrical response of the retina to a flash of light, as measured by electroretinogram [Time Frame: 1 month] Percent suppression compared to baseline of rod b-wave amplitude recovery after a photobleaching light.

This study may include three treatment groups. For example, a total of approximately 75 subjects may be enrolled and at the completion of the study, there may be approximately 25 subjects in each of the three treatment groups: 1) single evening dose 2) morning and evening dose and 3) placebo.

All subjects may receive a morning dose (either active or placebo) and an evening dose (either active or placebo) during the entire duration of treatment. For example, two dosing schedules of (S)-3-amino-4-(difluoromethylenyl)cyclopent-1-ene-1-carboxylic acid may be tested: a single evening dose (o.d.) and a morning plus evening dose (b.i.d) designed to provide a more sustained exposure. Schedule C is morning and evening placebo. All subjects may be up-titrated to the target dose unless this target dose is not tolerated (titration conventions described below). All subjects may receive treatment for a maximum of 12 weeks at their optimal tolerated dose.

Doses may be progressively increased in 0.02 mg/kg/day increments per week (active or placebo) to a target dose (in the evening) of 0.14 mg/kg/day, and morning dose. Each dose escalation may be performed after adequate tolerability has been assessed by caregiver and investigator. For example, treatment initiation at Day 1 with 1 dose, e.g., 0.02 mg/kg (active (Act) or placebo (Plc)) in the evening. Then target up-titration may begin at Day 3 (window+2 days): If no adverse event (AE) related to the study drug is observed by caregiver and/or the investigator, another dose (active or placebo) is added in the evening. Again at Day 7 (window+2 days), Day 10 (window+2 days and Day 14 (window+2 days) if no AE related to the study drug is observed by caregiver and/or the investigator, another dose (active or placebo) may be added in the morning.

Slowed up-titration or delayed up titration will be acceptable if tolerability does not allow immediate further dose-escalation at any of the above detailed days (3, 7, 10, 14). Down-titration in the case tolerability is not acceptable after a previous up-titration step or during the course of the 12 week treatment, dose can be reduced to the previous level or even further. However, once a tolerable dose has been reached, it shall remain constant for the duration of the treatment period. Once a target dose is achieved the treatment may continue. For example, at Day 14: Earliest day the target dose can be reached the subject may be kept stable until End of Treatment visit (week 12) unless intolerability requires down-titration.

All subjects will be screened for participation in the study up to 28 days prior to the first dose administration. Inclusion criteria may include one or more of the following: (1) Age≥18 years, ≤40 years; (2) Must possess a clinical diagnosis of Stargardt disease in one or both eyes and at least two pathogenic mutations of the ABCA4 gene. Descriptive statistics may be used to summarize all primary and secondary endpoints as well as baseline variables, by treatment group. For continuous variables, n, number of missing values, mean, standard deviation, median, minimum, and maximum will be provided. For categorical variables, frequency and percentage will be presented for each category. Confidence intervals (CI) will be provided where meaningful. All CIs will be two-sided 95% confidence intervals.

### Example 2

Prospective Assessment of the Safety and Efficacy of (1 S,3 S)-3-amino-4-(difluoromethylidene)cyclopentane-1-carboxylic Acid in Patients with Stargardt Disease

This study is designed to determine whether (1S,3S)-3-amino-4-(difluoromethylidene)cyclopentane-1-carboxylic acid leads to an improvement in Stargardt Disease. The primary objective of this study may be to evaluate the safety and tolerability from Baseline to Week 6 and Week 12 of (1S,3S)-3-amino-4-(difluoromethylidene)cyclopentane-1-carboxylic acid in subjects with Stargardt Disease across different dose levels and in two dosing schedules. The following dosing schedules may be tested against placebo: (1) Once daily (o.d.): An evening dose; and (2) Twice daily (b.i.d.): Evening and morning doses titrated to target doses.

The safety endpoints that relate to this study may include: (1) Frequency and severity of adverse events (AEs) and serious adverse events; (2) Vital signs (weight, blood pressure, temperature); (3) Laboratory parameters (electrolytes, lipids, glucose, liver and pancreas function tests, hematology, creatinine); (4) Suicidality assessed by ABC-Irritability Subscale; (5).

The secondary objective of this study may include the identification of a set of parameters that may best characterize the efficacy of (1 S,3 S)-3-amino-4-(difluoromethylidene)cyclopentane-1-carboxylic acid in Stargardt Disease subjects for subsequent efficacy trials. These tests may be administered at four full day site visits (Screening, Baseline, Interim and End of Treatment) by an appropriately trained professional to provide the test to an adult Stargardt Disease patient. Assessments may be based in part on patient's perception of symptoms. Secondary outcome measures also involve 1. Spectral Domain-Optical Coherence Tomography (SD-OCT) [Time Frame: 6 months] Mean rate of change in the area of ellipsoid zone defect measured by en face SD-OCT and 2. Change in electrical response of the retina to a flash of light, as measured by electroretinogram [Time Frame: 1 month] Percent suppression compared to baseline of rod b-wave amplitude recovery after a photobleaching light.

This study may include three treatment groups. For example, a total of approximately 75 subjects may be enrolled and at the completion of the study, there may be approximately 25 subjects in each of the three treatment groups: 1) single evening dose 2) morning and evening dose and 3) placebo.

All subjects may receive a morning dose (either active or placebo) and an evening dose (either active or placebo) during the entire duration of treatment. For example, two dosing schedules of (1S,3S)-3-amino-4-(difluoromethylidene)cyclopentane-1-carboxylic acid may be tested: 1. a single evening dose (o.d.) and, 2. a morning plus evening dose (b.i.d) designed to provide a more sustained exposure. Schedule C is morning and evening placebo. All subjects may be up-titrated to the target dose unless this target dose is not tolerated (titration conventions described below). All subjects may receive treatment for a maximum of 12 weeks at their optimal tolerated dose.

Doses may be progressively increased in 0.2 mg·kg/day increments per week (active or placebo) to a target dose of 1.2 mg/kg/day. Each dose escalation may be performed after adequate tolerability has been assessed by caregiver and investigator. For example, treatment initiation at Day 1 with 1 dose, e.g., 0.2 mg/kg (active (Act) or placebo (Plc)) in the evening. Then target up-titration may begin at Day 3 (window+2 days): If no adverse event (AE) related to the study drug is observed by caregiver and/or the investigator, another dose (active or placebo) is added. Again at Day 7 (window+2 days), Day 10 (window+2 days and Day 14 (window+2 days) if no AE related to the study drug is observed by caregiver and/or the investigator, another dose (active or placebo) may be added in the morning.

Slowed up-titration or delayed up titration will be acceptable if tolerability does not allow immediate further dose-escalation at any of the above detailed days (3, 7, 10, 14). Down-titration in the case tolerability is not acceptable after a previous up-titration step or during the course of the 12 week treatment, dose can be reduced to the previous level or even further. However, once a tolerable dose has been reached, it shall remain constant for the duration of the treatment period. Once a target dose is achieved the treatment may continue. For example, at Day 14: Earliest day the target dose can be reached the subject may be kept stable until End of Treatment visit (week 12) unless intolerability requires down-titration.

All subjects will be screened for participation in the study up to 28 days prior to the first dose administration. Inclusion criteria may include one or more of the following: (1) Age≥18 years, ≤40 years; (2) Must possess a clinical diagnosis of Stargardt disease in one or both eyes and at least two pathogenic mutations of the ABCA4 gene. Descriptive statistics may be used to summarize all primary and secondary endpoints as well as baseline variables, by treatment group. For continuous variables, n, number of missing values, mean, standard deviation, median, minimum, and maximum will be provided. For categorical variables, frequency and percentage will be presented for each category. Confidence intervals (CI) will be provided where meaningful. All CIs will be two-sided 95% confidence intervals.

### Example 3

### Prospective Assessment of the Development of Geographic Atrophy with (1 S,3 S)-3-amino-4-(difluoromethylidene)cyclopentane-1-carboxylic Acid in Patients with Wet Age Related Macular Degeneration

This will be a phase randomised, controlled, masked study recruiting patients with one treatment-naïve eye presenting with subfoveal choroidal neovascularisation (CNV) secondary to wet AMD. At screening, following the informed consent process, patients will be assessed for study eligibility by assessment of visual acuity using logarithm of the minimum angle of resolution (logMAR), stereoscopic biomicroscopic slit-lamp fundus examination (78 D or similar lens); fluorescein angiogram (FA); color fundus photography; fundus autofluorescence and optical coherence tomography (OCT). Eligible patients will be enrolled and then randomised to one of two arms: Arm 1: 1.0 mg (1 S,3S)-3-amino-4-(difluoromethylidene)cyclopentane-1-carboxylic acid using an inject and extend regimen; Arm 2: placebo.

Inclusion criteria for patients will be diagnosis of active subfoveal CNV secondary to wet AMD without restriction of lesion size, with visual impairment being exclusively due to an active wet AMD lesion. Active lesions will be characterized by any of the following: abnormal retinal thickness, with evidence of intraretinal, subretinal or sub-pigment epithelial fluid accumulation, confirmed by OCT; presence of intraretinal or subretinal haemorrhage; leakage shown on a FA unless solely due to dry, fibrotic staining; visual acuity deterioration considered likely to represent CNV. In addition, BCVA score at both Screening and Baseline must be 23 letters or more as measured by the 3 meter Early Treatment Diabetic Retinopathy Study (ETDRS)-like charts, inclusively (or approximate Snellen equivalent to 3/60+3).

Patients will receive 1.0 mg (1S,3S)-3-amino-4-(difluoromethylidene)cyclopentane-1-carboxylic acid via intravitreal injection.

Primary outcome measures are the mean change in area of geographic atrophy in the study eye from baseline to month 24 [Time Frame: Month 24] Measured by multimodal imaging assessed by an independent reading center masked (blinded) to the treatment arms.

Secondary outcome measures are Number of injections [Time Frame: Month 12 and 24]; Mean change in Best Corrected Visual Acuity (BCVA) (logMAR) [Time Frame: Month 12 and 24]; Mean change in area of existing and newly developed geographic atrophy [time Frame: Month 12]; Measured by multimodal imaging assessed by an independent reading center masked (blinded) to the treatment arms; Proportion of patients showing geographic atrophy [Time Frame: Month 12]; Mean change in central retinal thickness (CRT) [Time Frame: Months 12 and 24]; Proportion of patients showing no intraretinal fluid (IRF) [Time Frame: Months 2, 12 and 24]; Proportion of patients showing greater than and equal to a 15 letters gain [Time Frame: Months 12 and 24]; Proportion of patients showing less than and equal to a 15 letters loss [Time Frame: Months 12 and 24]; Number of times a patient needed to return to monthly treatments [Time Frame: Month 24]; Change in Retinal Nerve Fiber Thickness [Time Frame: Month 24]; Plasma VEGF levels Time Frame: Month 1 & 2]; Ocular and systemic adverse events [Time Frame: Month 24]; Ocular inflammation [Time Frame: After 3rd injection].

### Example 4

### Prospective Assessment of the Development of Geographic Atrophy with (S)-3-amino-4-(difluoromethylenyl)cyclopent-1-ene-1-carboxylic Acid in Patients with Wet Age Related Macular Degeneration

This will be a phase randomised, controlled, masked study recruiting patients with one treatment-naïve eye presenting with subfoveal choroidal neovascularisation (CNV) secondary to wet AMD. At screening, following the informed consent process, patients will be assessed for study eligibility by assessment of visual acuity using logarithm of the minimum angle of resolution (logMAR), stereoscopic biomicroscopic slit-lamp fundus examination (78 D or similar lens); fluorescein angiogram (FA); color fundus photography; fundus autofluorescence and optical coherence tomography (OCT). Eligible patients will be enrolled and then randomised to one of two arms: Arm 1: 0.5 mg (S)-3-amino-4-(difluoromethylenyl)cyclopent-1-ene-1-carboxyli c acid using an inject and extend regimen; Arm 2: placebo.

Inclusion criteria for patients will be diagnosis of active subfoveal CNV secondary to wet AMD without restriction of lesion size, with visual impairment being exclusively due to an active wet AMD lesion. Active lesions will be characterized by any of the following: abnormal retinal thickness, with evidence of intraretinal, subretinal or sub-pigment epithelial fluid accumulation, confirmed by OCT; presence of intraretinal or subretinal haemorrhage; leakage shown on a FA unless solely due to dry, fibrotic staining; visual acuity deterioration considered likely to represent CNV. In addition, BCVA score at both Screening and Baseline must be 23 letters or more as measured by the 3 meter Early Treatment Diabetic Retinopathy Study (ETDRS)-like charts, inclusively (or approximate Snellen equivalent to 3/60+3).

Patients will receive 0.5 mg (S)-3-amino-4-(difluoromethylenyl)cyclopent-1-ene-1-carboxylic acid via intravitreal injection.

Primary outcome measures are the mean change in area of geographic atrophy in the study eye from baseline to month 24 [Time Frame: Month 24] Measured by multimodal imaging assessed by an independent reading center masked (blinded) to the treatment arms.

Secondary outcome measures are Number of injections [Time Frame: Month 12 and 24]; Mean change in Best Corrected Visual Acuity (BCVA) (logMAR) [Time Frame: Month 12 and 24]; Mean change in area of existing and newly developed geographic atrophy [time Frame: Month 12]; Measured by multimodal imaging assessed by an independent reading center masked (blinded) to the treatment arms; Proportion of patients showing geographic atrophy [Time Frame: Month 12]; Mean change in central retinal thickness (CRT) [Time Frame: Months 12 and 24]; Proportion of patients showing no intraretinal fluid (IRF) [Time Frame: Months 2, 12 and 24]; Proportion of patients showing greater than and equal to a 15 letters gain [Time Frame: Months 12 and 24]; Proportion of patients showing less than and equal to a 15 letters loss [Time Frame: Months 12 and 24]; Number of times a patient needed to return to monthly treatments [Time Frame: Month 24]; Change in Retinal Nerve Fiber Thickness [Time Frame: Month 24]; Plasma VEGF levels Time Frame: Month 1 & 2]; Ocular and systemic adverse events [Time Frame: Month 24]; Ocular inflammation [Time Frame: After 3rd injection].

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method of treating an eye disorder comprising administering to a patient in need thereof (1 S,3 S)-3-amino-4-(difluoromethylidene)cyclopentane-1-carboxylic acid or a pharmaceutically acceptable salt thereof in an amount of from 0.01 mg to 500 mg, wherein the method provides improvement in one or more symptoms of eye disorder in the patient.

2. The method of claim 1, wherein the improvement is provided for more than 6 hours after administration.

3. The method of claim 1, wherein the patient is administered a composition comprising about 1 mg to 100 mg of (1S,3S)-3-amino-4-(difluoromethylidene)cyclopentane-1-carboxylic acid or a pharmaceutically acceptable salt thereof.

4. The method of claim 1, wherein the total amount of (1S,3S)-3-amino-4-(difluoromethylidene)cyclopentane-1-carboxylic acid or a pharmaceutically acceptable salt thereof administered to the subject in a twenty-four hour period is between 1 mg and 500 mg.

5. The method of claim 1, wherein (1S,3S)-3-amino-4-(difluoromethylidene)cyclopentane-1-carboxylic acid or a pharmaceutically acceptable salt thereof is administered from one to four times a day.

6. The method of claim 1, wherein administering is accomplished via a route selected from the group consisting of oral, buccal, sublingual, rectal, topical, intranasal, ophthalmic, vaginal and parenteral.

7. The method of claim 1, wherein the eye disorder is Stargardt disease, age-related macular degeneration, juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, choroideremia, Coats disease, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, or Wagner syndrome.

8. The method of claim 1, wherein the method provides improvement in at least one symptom selected from the group consisting vision loss, drusen amount, pigment changes in the retina, abnormal blood vessel growth, leaky blood vessels, macular swelling, corneal swelling, corneal thinning, accumulation of lipofuscin, night blindness, distorted vision, blurry vision, rod damage, cone damage, uvea inflammation, eye redness, pain, sensitivity to light (photophobia), floaters, eye flashes, nodules, orbital inflammation, lacrimal gland enlargement, decreased visual acuity, decrease in contrast sensitivity, blind spots, loss of color perception, loss of peripheral vision, fluid build-up in the macula, retinal scarring, double vision, pigment clumps, tunnel vision, thin cornea, spotting, leukocoria, lesions, crystals, and nystagmus.

9. A method of treating an eye disorder comprising administering to a subject with an eye disorder (S)-3-amino-4-(difluoromethylenyl)cyclopent-1-ene-1-carboxylic acid or a pharmaceutically acceptable salt thereof in an amount of from 0.01 mg to 75 mg.

10. The method of claim 9, wherein the subject is administered from 0.1 mg to 50 mg of (S)-3-amino-4-(difluoromethylenyl)cyclopent-1-ene-1-carboxylic acid or a pharmaceutically acceptable salt thereof.

11. The method of claim 9, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 10 mg.

12. The method of claim 9, wherein (S)-3-amino-4-(difluoromethylenyl)cyclopent-1-ene-1-carboxylic acid or a pharmaceutically acceptable salt thereof is administered from one to four times a day.

13. The method of claim 9, wherein administering is accomplished via a route selected from the group consisting of oral, buccal, sublingual, rectal, topical, intranasal, ophthalmic, vaginal and parenteral.

14. The method of claim 9 wherein the eye disorder is Stargardt disease, age-related macular degeneration, juvenile macular degeneration, retinal degeneration, glaucoma, retinal dystrophy, Doyne honeycomb retinal dystrophy, light induced retinal damage, uveitis, scleritis, ocular sarcoidosis, optic neuritis, cone-rod dystrophy, macular edema, diabetic retinopathy, diabetic macular edema, corneal ulcer, an autoimmune disorder, ophthalmic manifestations of AIDS, optic nerve degeneration, geographic atrophy, choroidal dystrophy, retinitis, CMV retinitis, reticular pseudodrusen, eye floaters, eye flashes, keratoconus, ocular hypertension, presbyopia, dry eyes, Bietti's Crystalline Dystrophy, retinoblastoma, Usher syndrome, Behçet's disease, Achromatopsia 2, acute posterior multifocal placoid pigment epitheliopathy, acute zonal occult outer retinopathy, adult-onset vitelliform macular dystrophy, ocular albinism with late-onset sensorineural deafness, Alström syndrome, anterior ischemic optic neuropathy, corneal amyloidosis, gelatinous drop-like corneal dystrophy, Axenfeld-Rieger syndrome, Bardet-Biedl syndrome, Behr syndrome, Best disease aka vitelliform macular dystrophy, Bietti crystalline corneoretinal dystrophy, birdshot chorioretinopathy, blue cone monochromatism, central areolar choroidal dystrophy, iridocorneal endothelial syndrome, Avellino type corneal dystrophy, Schnyder corneal dystrophy, Thiel-Behnke corneal dystrophy, Eales disease, epithelial basement membrane corneal dystrophy, Fish-eye disease, Fuchs endothelial corneal dystrophy, Goldmann-Favre syndrome, juvenile retinoschisis, late-onset retinal degeneration, Leber congenital amaurosis, retinitis pigmentosa, Peters anomaly, punctate inner choroidopathy, Senior Loken syndrome, snowflake vitreoretinal degeneration, Usher syndrome, visual snow syndrome, or Wagner syndrome.

15. The method of claim 9, wherein the method provides improvement in at least one symptom selected from the group consisting of vision loss, drusen amount, pigment changes in the retina, abnormal blood vessel growth, leaky blood vessels, macular swelling, corneal swelling, corneal thinning, accumulation of lipofuscin, night blindness, distorted vision, blurry vision, rod damage, cone damage, uvea inflammation, eye redness, pain, sensitivity to light (photophobia), floaters, eye flashes, nodules, orbital inflammation, lacrimal gland enlargement, decreased visual acuity, decrease in contrast sensitivity, blind spots, loss of color perception, loss of peripheral vision, fluid build-up in the macula, retinal scarring, double vision, pigment clumps, tunnel vision, thin cornea, spotting, leukocoria, lesions, crystals, and nystagmus.

16. A method of treating Stargardt disease comprising administering to a patient in need thereof (1 S,3 S)-3-amino-4-(difluoromethylidene)cyclopentane-1-carboxylic acid or a pharmaceutically acceptable salt thereof in an amount of from 0.01 mg to 500 mg, wherein the method provides improvement in one or more symptoms of Stargardt disease in the patient.

17. The method of claim 16, wherein the improvement is provided for more than 6 hours after administration.

18. The method of claim 16, wherein the patient is administered a composition comprising about 1 mg to 100 mg of (1S,3S)-3-amino-4-(difluoromethylidene)cyclopentane-1-carboxylic acid or a pharmaceutically acceptable salt thereof.

19. The method of claim 16, wherein the total amount of (1S,3S)-3-amino-4-(difluoromethylidene)cyclopentane-1-carboxylic acid or a pharmaceutically acceptable salt thereof administered to the subject in a twenty-four hour period is between 1 mg and 500 mg.

20. The method of claim 16, wherein (1S,3S)-3-amino-4-(difluoromethylidene)cyclopentane-1-carboxylic acid or a pharmaceutically acceptable salt thereof is administered from one to four times a day.

21. The method of claim 16, wherein administering is accomplished via a route selected from the group consisting of oral, buccal, sublingual, rectal, topical, intranasal, ophthalmic, vaginal and parenteral.

22. The method of claim 16, wherein the method provides improvement in at least one symptom selected from the group consisting of macular degeneration, vision loss, accumulation of lipofuscin in the macula, night blindness, and loss of color vision.

23. A method of treating Stargardt disease comprising administering to a patient in need thereof (S)-3-amino-4-(difluoromethylenyl)cyclopent-1-ene-1-carboxylic acid or a pharmaceutically acceptable salt thereof in an amount of from 0.01 mg to 500 mg, wherein the method provides improvement in one or more symptoms of Stargardt disease in the patient.

24. The method of claim 23, wherein the improvement is provided for more than 6 hours after administration.

25. The method of claim 23, wherein the patient is administered a composition comprising about 0.01 mg to 50 mg of (1 S,3 S)-3-amino-4-(difluoromethylidene)cyclopentane-1-carboxylic acid or a pharmaceutically acceptable salt thereof.

26. The method of claim 23, wherein the total amount of (S)-3-amino-4-(difluoromethylenyl)cyclopent-1-ene-1-carboxylic acid or a pharmaceutically acceptable salt thereof administered to the subject in a twenty-four hour period is between 1 mg and 500 mg.

27. The method of claim 23, wherein (S)-3-amino-4-(difluoromethylenyl)cyclopent-1-ene-1-carboxylic acid or a pharmaceutically acceptable salt thereof is administered from one to four times a day.

28. The method of claim 23, wherein administering is accomplished via a route selected from the group consisting of oral, buccal, sublingual, rectal, topical, intranasal, ophthalmic, vaginal and parenteral.

29. The method of claim 23, wherein the method provides improvement in at least one symptom selected from the group consisting of macular degeneration, vision loss, accumulation of lipofuscin in the macula, night blindness, and loss of color vision.
